(19) United States
(12) Patent Application Publication
(10) Pub. No.: US 2026/0306516 A1
(43) Pub. Date: Oct. 8, 2026

(54) CANNABINOID-BASED FORMULATION

(71) Applicant: MRX Medical Limited, Weybridge (GB)

(72) Inventors: Charles MORGAN, London (GB); Melissa STURGESS, London (GB); Nicholas CLARKSON, London (GB)

(21) Appl. No.: 19/483,070

(22) PCT Filed: May 10, 2024
(86) PCT No.: PCT/GB2024/051227

§ 371 Date: Nov. 10, 2025

(30) Foreign Application Priority Data
May 11, 2023 (GB) 2307002.2

(51) Int. Cl. A61K 31/00; A61K 31/01; A61K 31/015; A61K 31/045; A61K 47/14; A61P 25/02; A61P 29/00

(52) U.S. Cl. CPC A61K 31/658; A61K 31/01; A61K 31/015; A61K 31/045; A61K 47/14; A61P 25/02; A61P 29/00

(57) ABSTRACT

The invention relates to cannabinoid-based formulations, and particularly, although not exclusively, to methods for preparing cannabinoid-based formulations, and to the formulations per se, and their use in therapy. The invention is especially concerned with cannabidiol (CBD)-based formulations, their manufacture and uses, for treating pain, inflammation, autoimmune diseases, neurological diseases, neoplasm, nausea, hypertension, hypertrophic cardiomyopathy, or fibrosis.

The present invention relates to cannabinoid-based formulations, and particularly, although not exclusively, to methods for preparing cannabinoid-based formulations, and to the formulations per se, and their use in therapy. The invention is especially concerned with cannabidiol (CBD)-based formulations, their manufacture and uses, for treating pain, inflammation, autoimmune diseases, neurological diseases, neoplasm, nausea, hypertension, hypertrophic cardiomyopathy, or fibrosis.

The cannabinoids are several structural classes of compounds found primarily in the cannabis plant. The best studied cannabinoids include cannabidiol (CBD), tetrahydrocannabinol (THC) and cannabinol (CBN), with CBD being the most widely investigated phytocannabinoid (i.e., a plant-derived cannabinoid) due to its therapeutic activities and lack of psychoactive effects. In addition, this compound has shown a large variety of pharmacological effects, such as anti-inflammatory, analgesic and anxiolytic properties, and has been used alone for multiple health conditions for many years.

In addition to the pharmaceutical activity of cannabinoids, such as CBD, recent evidence has suggested that terpenes (responsible for the aromas and flavours of many plants) which are also found in the Cannabis sativa plant and other plants in varying amounts and types are also pharmacologically active. Terpenes can, in some instances, bind to the same receptors as cannabinoids, as well as having distinct beneficial actions themselves, and can produce an entourage effect by modulating cannabinoids to result in improved outcomes over either chemical alone. A possible mechanism of the synergistic activity between cannabinoids and terpenes is by promoting the activated form of the cannabinoid receptors, modulation of endocannabinoid synthesis and degradation, and subsequent receptor activation by these endocannabinoids. Additionally, terpenes have been proven to enhance drug permeation via various mechanisms explained in detail elsewhere.

Unfortunately, the CBD that is used for the treatment of various health conditions is usually extracted directly from the cannabis plant, and it is very difficult, if not impossible, to standardise CBD extracts due to a wide variety of factors influencing the biochemical composition of the cannabis plant, such as the geographical location of where the cannabis plant is grown, the fluctuating growth conditions of the plant (i.e. soil and weather conditions), the time of year of CBD extraction from the plant, and differences in extraction methods from the plant, etc. This significant variability in the cannabis plant and extraction parameters results in the CBD formulations that are currently available suffering from the problem of not being consistently reproducible and so not always having the correct amounts of CBD that is listed on the label (Liebling et al., 2022, An Analysis of Over-the-Counter Cannabidiol Products in the United Kingdom, Cannabis and Cannabinoid Research, 7, 2, 207-213).

Furthermore, cannabis plants can have widely different levels and composition of terpenes, which, as mentioned above, are believed to confer a wide range of beneficial activities due to the entourage effect they create by modulating the cannabinoids. However, the extraction methods that are currently used also do not allow the terpenes to be extracted from the cannabis plant in a reproducibly consistent way, and so, as with the CBD, this lack of reproducibility and variability of terpenes between batches means that customers cannot be certain that they are taking exactly the same CBD/terpene mix to treat their condition. This variability in the composition of the CBD formulation from batch to batch results in differences in the therapeutic activity between the batches of the formulation. Moreover, there have been examples where moving from one formulation back to another has had very detrimental effects on patients' health (Booth and Bohlmann, 2019, Terpenes in Cannabis sativa—From plant genome to humans, 284, July, 67-72).

Another problem with currently available CBD formulations is that the direct extraction of CBD from the natural cannabis plant frequently results in differing levels of tetrahydrocannabinol (THC) contamination. THC is a controlled drug and has unwanted addictive and psychotropic activities that are not required for the beneficial activities of CBD for treatment of the various conditions listed above. There are, therefore, strict legal limits on how much THC can be present in a CBD formulation. However, there are safety issues with THC, and current extraction methods mean it cannot be removed completely when the CBD and terpenes are extracted from the cannabis plant, meaning that CBD products are often contaminated with THC (Liebling et al., 2022, An Analysis of Over-the-Counter Cannabidiol Products in the United Kingdom, Cannabis and Cannabinoid Research, 7, 2, 207-213).

Formulations comprising cannabinoids and terpenes are referred to as a Cannabis-Based Product for Medicinal use (CBPM). CBPMs have been added to the treatment paradigm for many patients suffering from acute and chronic inflammatory conditions, pain, and symptoms of negative affect, such as anxiety and depression that have limited or a lack of response to traditional therapeutic approaches. Due to this, the National Institute for Health and Care Excellence (NICE) and the International Association for the Study of Pain (IASP) have made specific calls for research using CBPMs in humans. However, there is still an unmet need for treating autoimmune diseases, multiple treatment-resistant conditions, such as fibromyalgia, chemotherapy-induced peripheral neuropathy (CIPN) and endometriosis, which are characterised by pain, inflammation and other negative effects that have an adverse influence on the quality of life of patients suffering from these conditions.

Since no one-size-fits-all therapy is currently available for the above-mentioned conditions, most patients require multiple and alternative treatment approaches that are often inconvenient and cause multiple side effects. Additionally, whilst numerous CBD formulations are now available as food supplements, making many beneficial health claims, there is minimal assessment of whether they are being administered in a suitable formulation even to achieve blood levels to support these claims, and a recent study analysing a range of CBD containing products in the UK found that there is a great variation in the levels of CBD actually present in these products with varying levels of THC contamination (Liebling et al., 2022, An Analysis of Over-the-Counter Cannabidiol Products in the United Kingdom, Cannabis and Cannabinoid Research, 7, 2, 207-213). However, to date, there are no CBD-containing products available that comply with GMP standards.

Accordingly, there is a need for a new medical CBPM formulation for the treatment of various conditions, such as pain, inflammation, autoimmune disease, neurological disease, neoplasm, nausea, hypertension, hypertrophic cardiomyopathy, and fibrosis, and which is produced under defined pharmaceutical standards and under the guidelines of GMP for use in medical research and practice, and in foodstuffs, and which therefore facilitates the access and delivery of a pharmaceutical standard CBPM to clinical trials and to benefit patients. There is also a need to produce standardised cannabinoid (e.g., CBD)-based formulations for treating these conditions which contain reproducibly consistent levels of the desired cannabinoid(s) as well as the associated terpenes while lacking any significant levels of THC. This standardisation means that the composition and biological activity of the CBD formulation will be the same between batches, giving patients significantly more confidence in the CBPM product and its therapeutic effects for treating pain, inflammation, autoimmune disease, neurological disease, neoplasm, nausea, hypertension, hypertrophic cardiomyopathy, and fibrosis.

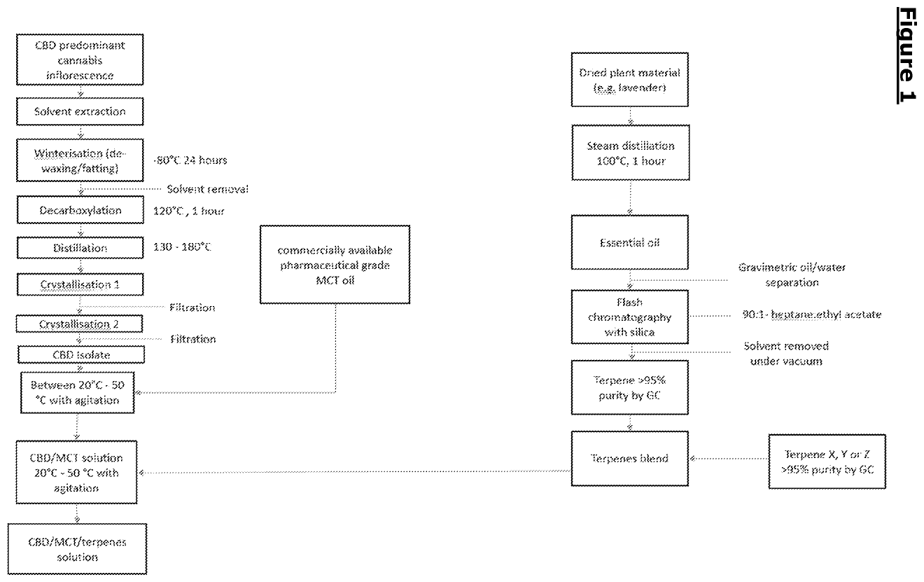

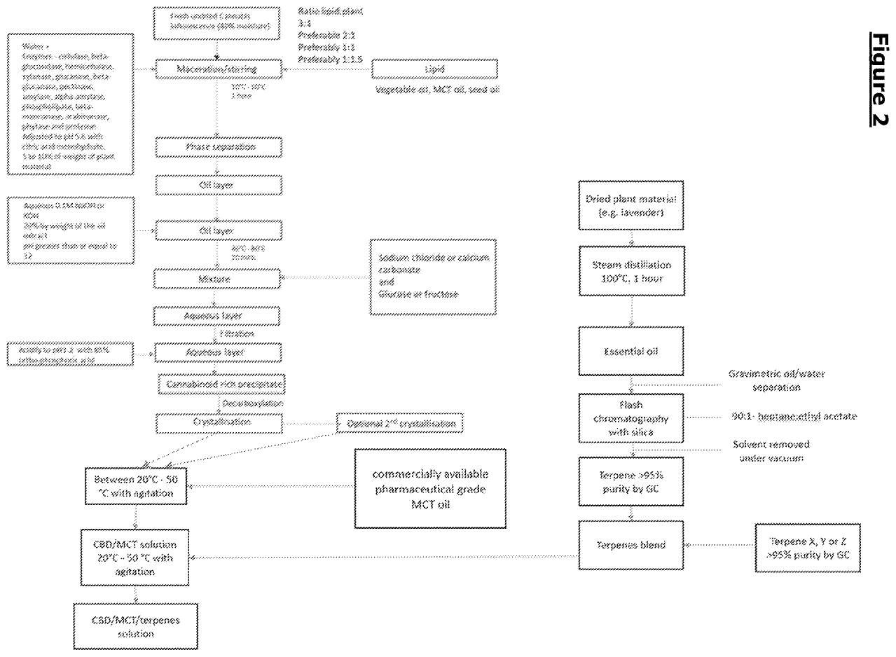

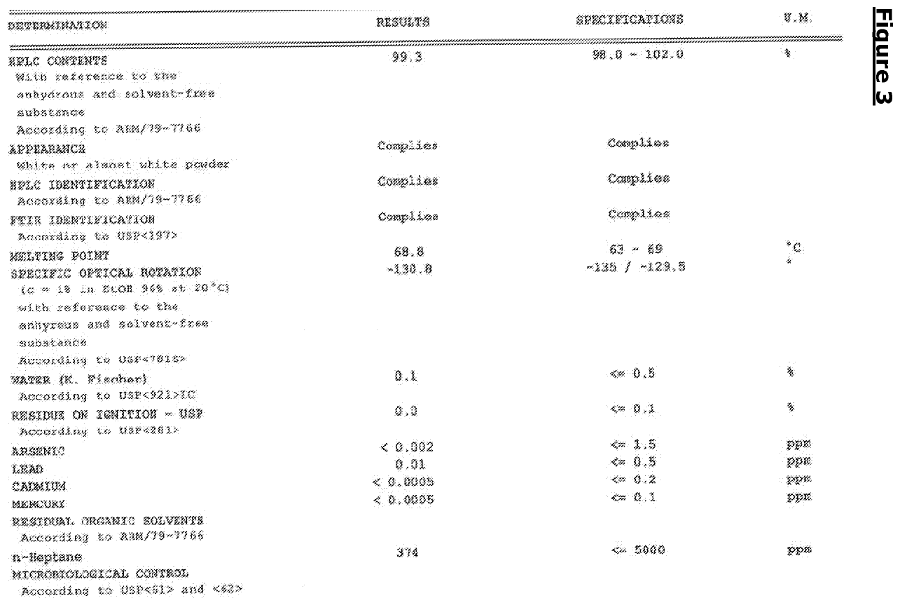

As described in the Examples, and as shown in FIGS. 1 and 2, the inventors have devised an innovative method for producing embodiments of a highly standardised CBD-based formulation, as shown in Tables 3, 7, and 8, for the treatment of pain, inflammation, autoimmune diseases, neurological diseases, neoplasm, nausea, hypertension, hypertrophic cardiomyopathy, and fibrosis. The formulation comprises the cannabinoid, CBD, and a highly specific mixture of terpenes from scratch using an isolate of CBD of known purity and a highly defined mix of terpenes allowing the preparation of a formulation with standardised amounts of each component, whilst simultaneously minimising any contamination with THC.

Thus, according to a first aspect of the invention, there is provided a cannabinoid-based formulation comprising:
(i) one or more cannabinoid; and
(ii) a plurality of terpenes selected from a group of terpenes consisting of: myrcene; caryophyllene; ocimene; pinene; limonene; humulene; and linalool;
   wherein the formulation substantially lacks THC.

In a second aspect, there is provided a method of producing the cannabinoid-based formulation according to the first aspect, the method comprises combining: (i) one or more cannabinoid; and (ii) a plurality of terpenes selected from a group of terpenes consisting of: myrcene; caryophyllene; ocimene; pinene; limonene; humulene; and linalool, to produce a cannabinoid-based formulation, wherein the formulation substantially lacks THC.

In a third aspect, there is provided a cannabinoid-based formulation obtained or obtainable by the method of the second aspect.

In a fourth aspect, there is provided the cannabinoid-based formulation of the first or third aspect, for use in therapy.

In a fifth aspect, there is provided the cannabinoid-based formulation of the first or third aspect, for use in the treatment, amelioration or prevention of pain, inflammation, an autoimmune disease, a neurological disease, neoplasm, nausea, hypertension, hypertrophic cardiomyopathy, or fibrosis.

In a sixth aspect, there is provided a method of treating, preventing or ameliorating a subject suffering from pain, inflammation, an autoimmune disease, a neurological disease, neoplasm, nausea, hypertension, hypertrophic cardiomyopathy, or fibrosis, the method comprising administering, or having administered, to a subject in need of such treatment, a therapeutically active amount of the cannabinoid-based formulation of the first or third aspect.

Advantageously, and preferably, the method of the second aspect combines the one or more cannabinoid of a known purity, and the plurality of terpenes forming a mixture, also of known purity, to produce the formulation of the first or third aspect which is fully standardized with a known concentration and purity of each of the constituents, and is fully reproducible, with no variability between different batches. It will be appreciated that the cannabinoid-based formulation of the invention is referred to herein as MRX1. One embodiment of the formulation (i.e., “MRX1”) is shown in Table 3. Accordingly, a certificate of analysis can be given for each batch of the formulation of the invention using the method of the second aspect, showing its stability data, as well as evidence that there is substantially no contamination with any THC, thereby providing the patient with full confidence of the composition of the formulation and, therefore, its therapeutic efficacy.

THC may be detected using any standard analytical chemistry approaches, such as liquid chromatography with ultraviolet (UV) or mass spectrometric (MS or MS/MS) detection, e.g. Lukas Vaclavik et al., (2019) ‘Quantitation of Cannabinoids in Cannabis Dried Plant Materials, Concentrates, and Oils Using Liquid Chromatography-Diode Array Detection Technique with Optional Mass Spectrometric Detection: Single-Laboratory Validation Study, First Action 2018.11’ Journal of AOAC INTERNATIONAL, Volume 102, Issue 6, 1 Nov. 2019, Pages 1822-1833.

The concentration of THC in the formulation of the first or third aspect is preferably less than 1000 μg per finished product, e.g. for 30 ml this equates to 0.0033% (w/v). Therefore, preferably the concentration of THC in the formulation is less than 0.01% (w/v), more preferably less than 0.005% (w/v), and even more preferably less than 0.001% (w/v). Preferably, the concentration of THC in the formulation is less 0.0005% (w/v), and even more preferably less than 0.0002% (w/v), and most preferably less than 0.0001% (w/v). Preferably, however, the formulation comprises no detectable THC using reasonable validated means of analysis.

The term ‘cannabinoid’ can refer to any lipophilic ligand capable of interacting with receptors involved in the endocannabinoid system (ECS). The cannabinoid may comprise a phytocannabinoid (i.e., plant cannabinoid), an endocannabinoid (i.e. endogenous cannabinoids) or a synthetic cannabinoid (i.e. man-made). Traditionally, cannabinoids were thought to bind selectively to the cannabinoid receptors (CB) 1 and 2. However, further research into ECS pharmacology has led to the discovery of various molecular targets for cannabinoids independent of the CBRs 1 and 2, such as the transient receptor potential (TRP) channels and two orphan GPCRs G-protein-coupled receptors (GPR55/18). Chemically, cannabinoids are meroterpenoids (C₂₁₋₂₂ terpenophenolic compounds) derived from the alkylation of an alkyl resorcinol with a monoterpene unit. They are mainly synthesised and stored in glandular trichomes, most abundant in female inflorescences of the cannabis plant.

The one or more cannabinoid may be selected from a group of cannabinoids consisting of: cannabidiol (CBD); cannabichromene (CBC); cannabigerol (CBG); and cannabigerol monomethyl ether (CBGM). The one or more cannabinoid may comprise two or more cannabinoids selected from a group consisting of: cannabidiol (CBD); cannabichromene (CBC); cannabigerol (CBG); and cannabigerol monomethyl ether (CBGM). The one or more cannabinoid may comprise three or more cannabinoids selected from a group consisting of: cannabidiol (CBD); cannabichromene (CBC); cannabigerol (CBG); and cannabigerol monomethyl ether (CBGM). The one or more cannabinoid may comprise four or more cannabinoids selected from a group consisting of: cannabidiol (CBD); cannabichromene (CBC); cannabigerol (CBG); and cannabigerol monomethyl ether (CBGM).

Preferably, however, the one or more cannabinoid comprises CBD. The one or more cannabinoid may comprise an isolate of CBD. Preferably, the formulation does not comprise CBC. Preferably, the formulation does not comprise CBG. Preferably, the formulation does not comprise CBGM.

Preferably, the chemical structure of the phytocannabinoid, CBD, used in the method of the invention, comprises Formula [I]:—

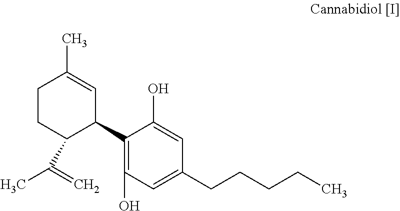 [SMILES: C=C(C)[C@@H]1CCC(C)=C[C@H]1c1c(O)cc(CCCCC)cc1O]

With reference to FIG. 1, in one embodiment, the one or more cannabinoid may be prepared with the use of a solvent (e.g. alcohol or alkane) extraction from plant material (e.g. inflorescence and/or leaves of Cannabis sativa), producing a slurry which may then be filtered to produce a crude liquid extract. The crude extract may then be chilled (to <−20° C. or <−40° C. for at least 12 hours, and preferably −80° C. for 24 hours) to precipitate fats and waxes that are subsequently filtered off to produce a ‘winterised’ extract. The solvent may then be removed under reduced pressure and the remaining crude oil is then preferably ‘decarboxylated’ to convert the acidic compounds to neutral forms, which is typically performed at >100° C. for at least 20 minutes, more preferably 120° C. for at least 30 minutes. The resulting decarboxylated extract may then be further purified by distillation to remove residual extraneous non-cannabinoid materials. The distillation step may be carried out at between 10° and 200° C., preferably between 130-180° C., and may be short path distillation. The resulting material may be dissolved in a solvent (e.g. alcohol) to around the saturation point of the solution, before an anti-solvent (e.g. alkane) is (slowly) added to induce crystallisation. Upon completion of the first crystallisation (~95% purity cannabinoid, preferably CBD), the solid material may then be filtered off and re-dissolved in alcohol, and a second crystallisation may be performed to produce an isolated cannabinoid (preferably, CBD) of the required specification.

With reference to FIG. 2, in a preferred embodiment, the one or more cannabinoid may be prepared by using enzyme digestion of plant material (e.g. inflorescence and/or leaves of Cannabis sativa). The plant material may comprise a moisture content of at least 20%, preferably at least 30%, more preferably at least 40%. Preferably, the plant material is contacted with an enzyme solution. The enzyme solution preferably comprises one or more enzyme selected from a group of enzymes consisting of: cellulase, beta-glucosidase, hemicellulase, xylanase, glucanase, beta-glucanase, pectinase, amylase, alpha-amylase, phospholipase, beta-mannanase, arabinanase, phytase and protease. Preferably, the pH of the enzyme solution is adjusted to at least about pH4, more preferably at least about pH5, and even more preferably about pH5.6. The pH adjustment may be achieved using citric acid. A weight ratio of 1:20 of aqueous enzyme solution relative to plant material may be used. Preferably, about 1-10% (wt) pf plant material is used to 99-10% (wt) enzyme solution. The plant material is preferably macerated before, during and/or after contacting with the enzyme solution.

Preferably, the plant material is contacted with a lipid for extracting plant compounds of interest (i.e., the cannabinoid). For example, the lipid may be vegetable oil, MCT oil, seed oil or olive oil. The weight ratio of lipid to plant material is about 2:1, preferably 1:1, more preferably 1:1.5. The plant material is preferably macerated before, during and/or after contacting with the lipid. The mixture is preferably stirred and macerated. This mixture is preferably agitated for at least 30, 40 or 60 minutes at a temperature of between about 5° C. and 80° C., more preferably between 8° C. and 65° C., and most preferably between 10° C. and 50° C. Upon full breakdown and dissolution of the plant material, the oil and water phases are preferably separated by gravity, centrifugation, filtration or a combination thereof. The oil layer is preferably transferred to a separate vessel having agitation capability. The oil layer is preferably contacted with an aqueous base. The base may be an aqueous solution of 0.1M NaOH or KOH, preferably at a pH greater than or equal to 12. The weight of aqueous basic solution may be 1:3 ratio by weight relative to the cannabinoid-containing oil, preferably 1:4, more preferably 1:5. This mixture is preferably agitated for at least 10-20 minutes at a temperature of between about 20° C. and 100° C., more preferably between 30° C. and 90° C., and most preferably between 40° C. and 80° C. On completion of the previous step, the mixture is preferably contacted with a combination of: (i) sodium chloride and/or calcium carbonate; and (ii) glucose and/or fructose. Preferably, this mixture is agitated for a further 10, 15 or 20 minutes.

Upon completion, the layers are preferably separated by gravity or centrifugation, and the aqueous layer may then be separated off. Preferably, the pH of the separated aqueous layer is reduced to a pH of less than 3, 2 or 1. This may be achieved by contacting the aqueous layer with acid, more preferably phosphoric acid (e.g. 85% ortho-phosphoric acid). Preferably, the solution is agitated for a further 10, 15 or 20 minutes, causing precipitation of cannabinoid acid salts.

Preferably, this liquid is filtered, giving a cannabinoid-rich solid precipitate between 65%-90% total cannabinoid content. The cannabinoid-rich precipitate may then be heated to at least 90, 100 or 120° C. for at least 30 mins, 45 mins or 60 mins, or until such time that complete decarboxylation has been achieved. The resulting material may be dissolved in a solvent (e.g., alcohol) to around the saturation point of the solution, before an anti-solvent (e.g., alkane) is (slowly) added to induce crystallisation. Optionally, the solid material may then be filtered off and re-dissolved in solvent (e.g., alcohol), and a second crystallisation may be performed to produce an isolated cannabinoid (preferably, CBD) of the required specification.

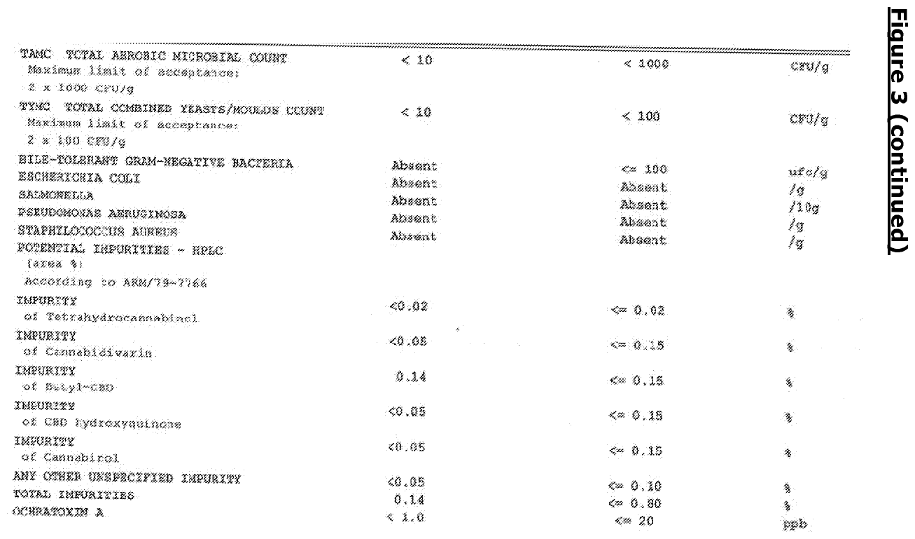

The purity of the one or more cannabinoid (preferably, CBD) may be at least 97%, at least 98% or at least 99%, or about 98-100%, as measured by HPLC-UV assay at 220 nm detection wavelength. As shown in FIG. 3, the inventors have a certificate of analysis (COA) for the CBD isolate. The specification has been set in order to comply with ICH Q3A guidance on impurities in new Drug Substance, i.e., to demonstrate sufficient control of Organic impurities (minor cannabinoids, solvents etc.), inorganic impurities (e.g., heavy metals) and microbiological contamination. Particularly relevant is the decision to set the specification for d9-tetrahydrocannabinol to 0.02%, which is lower than the requirements of less than, or equal to, 0.15% for any single known organic impurity.

The concentration of the one or more cannabinoid (preferably, CBD) in the cannabinoid-based formulation may be at least 2% (w/v), at least 3% (w/v) or at least 4% (w/v). The concentration of the one or more cannabinoid (preferably, CBD) in the cannabinoid-based formulation may be at least 5% (w/v), at least 6% (w/v) or at least 7% (w/v). Preferably, the concentration of the one or more cannabinoid (preferably, CBD) in the cannabinoid-based formulation may be at least 8% (w/v), at least 9% (w/v) or at least 10% (w/v). Preferably, the concentration of the one or more cannabinoid (preferably, CBD) in the cannabinoid-based formulation may be at least 11% (w/v), or at least 12% (w/v).

The concentration of the one or more cannabinoid (preferably, CBD) in the cannabinoid-based formulation may be less than 20% (w/v), less than 19% (w/v) or less than 18% (w/v). The concentration of the one or more cannabinoid (preferably, CBD) in the cannabinoid-based formulation may be less than 17% (w/v), less than 16% (w/v) or less than 15% (w/v). Preferably, the concentration of the one or more cannabinoid (preferably, CBD) in the cannabinoid-based formulation may be less than 14% (w/v), less than 13% (w/v) or less than 12% (w/v). Preferably, the concentration of the one or more cannabinoid (preferably, CBD) in the cannabinoid-based formulation may be less than 11% (w/v), less than 10% (w/v) or less than 9% (w/v).

The concentration of the one or more cannabinoid (preferably, CBD) in the cannabinoid-based formulation may be between 1 and 25% (w/v), between 3 and 20% (w/v) or between 5 and 17% (w/v). Preferably, the concentration of the one or more cannabinoid (preferably, CBD) in the cannabinoid-based formulation is between 7 and 13% (w/v), between 8 and 12% (w/v) or between 9 and 11% (w/v). Preferably, the concentration of the one or more cannabinoid (preferably, CBD) in the cannabinoid-based formulation is about 10% (w/v). It will be appreciated that any of the lower values for the concentration of the one or more cannabinoid mentioned herein may be combined with any of the upper values for the concentration of the one or more cannabinoid.

The plurality of terpenes is selected from a group of terpenes consisting of: myrcene; caryophyllene; ocimene; pinene; limonene; humulene; and linalool. Terpenes are lipids (complex compounds of fatty acids) and belong to the terpenoid family. Chemically, terpenes present a distinctive carbon skeleton which consists of a basic five-carbon isoprene unit (C₅H₈, 2-methyl-1,3-butadiene) linked together most commonly in a head-to-tail arrangement. However, they can be built in other configurations with varying degrees of oxidation, unsaturation, functional groups and ring closures, leading to a vast diversity of structural classes. Nevertheless, terpenes can be grouped into categories according to the number of isoprene units (n) in the molecule. Isoprene is a gaseous hydrocarbon released by multiple plants as a natural by-product of plant metabolism. However, some of the larger and more complex terpenes, such as squalene and lanosterol, are also found in animals.

As illustrated in FIGS. 1 and 2, the plurality of terpenes may be prepared by first steam distillation from the appropriate species of plant to produce an essential oil. The resulting essential oil may then be purified by chromatography (preferably by flash chromatography) to separate the individual terpene components at the required purity.

The purity of the plurality of terpenes may be at least 90%, preferably at least 92% and more preferably at least 95% of total terpenes as measured by GC-MS.

The plurality of terpenes may be selected from a group of terpenes shown in Table 1.

TABLE 1
Classification of Terpenes

| Classification | Isoprene units (n) | Carbon Atoms |
|---|---|---|
| Monoterpenes | 2 | 10 |
| Sesquiterpenes | 3 | 15 |
| Diterpenes | 4 | 20 |
| Sesterpenes | 5 | 25 |
| Triterpenes | 6 | 30 |
| Tetraterpenes | 8 | 40 |
| Polyterpenes | >100 | 50 |

Thus, the plurality of terpenes may be selected from a group of terpenes consisting of: monoterpenes, sesquiterpenes, diterpenes, sesterpenes, triterpenes, tetraterpenes and polyterpenes.

Preferably, the monoterpene is selected from a group of monoterpenes consisting of: Alpha-Pinene, Limonene, Cis-Ocimene, Beta-Myrcene and Linalool.

Preferably, the sesquiterpene is selected from a group of sesquiterpenes consisting of: Beta-Caryophyllene and Alpha-Humulene.

The chemical structure of the terpenes which may be present in the formulation are shown below. Terpenes are grouped depending on the carbon number.

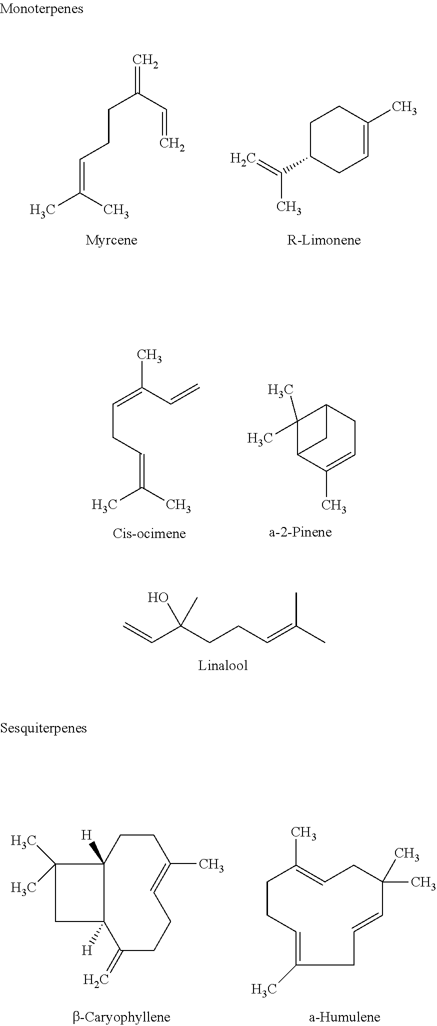

As described in the Examples, the inventors have synthesised a preferred embodiment of the cannabinoid-based formulation of the invention with a specific composition of terpenes. It also includes a cannabinoid (i.e., CBD), and an excipient or carrier (e.g., a MCT oil). In one embodiment, the composition is the formulation as summarised in Table 3, and its terpene mix is described below.

Preferably, the plurality of terpenes comprises at least three terpenes selected from a group of terpenes consisting of: myrcene; caryophyllene; ocimene; pinene; limonene; humulene; and linalool.

Preferably, the plurality of terpenes comprises at least four terpenes selected from a group of terpenes consisting of: myrcene; caryophyllene; ocimene; pinene; limonene; humulene; and linalool.

Preferably, the plurality of terpenes comprises at least five terpenes selected from a group of terpenes consisting of: myrcene; caryophyllene; ocimene; pinene; limonene; humulene; and linalool.

Preferably, the plurality of terpenes comprises at least six terpenes selected from a group of terpenes consisting of: myrcene; caryophyllene; ocimene; pinene; limonene; humulene; and linalool.

Preferably, the plurality of terpenes comprises at least seven terpenes selected from a group of terpenes consisting of: myrcene; caryophyllene; ocimene; pinene; limonene; humulene; and linalool.

Thus, in a preferred embodiment of the cannabinoid-based formulation of the invention, the plurality of terpenes comprises pinene, more preferably α-Pinene.

The concentration of α-Pinene in the cannabinoid-based formulation may be at least 0.0001 (w/v), or at least 0.001% (w/v). Preferably, the concentration of α-Pinene in the cannabinoid-based formulation may be at least 0.005% (w/v), at least 0.01% (w/v), or at least 0.02% (w/v). The concentration of α-Pinene in the cannabinoid-based formulation may be less than 0.75% (w/v), less than 0.5% (w/v) or less than 0.3% (w/v). The concentration of α-Pinene in the cannabinoid-based formulation may be less than 0.2% (w/v), less than 0.15% (w/v), or less than 0.1% (w/v). The concentration of α-Pinene in the cannabinoid-based formulation may be between 0.0001 and 0.5% (w/v), between 0.01 and 0.3% (w/v) or between 0.01 and 0.2% (w/v). The concentration of α-Pinene in the cannabinoid-based formulation may be between 0.01 and 0.05% (w/v), between 0.01 and 0.04% (w/v) or between 0.01 and 0.03% (w/v). Preferably, the concentration of α-Pinene in the cannabinoid-based formulation is about 0.02% (w/v). Preferably, the concentration of α-Pinene in the cannabinoid-based formulation is about 0.0225% (w/v). Preferably, the concentration of α-Pinene in the cannabinoid-based formulation is about 0.0230% (w/v). More preferably, the concentration of α-Pinene in the cannabinoid-based formulation is about 0.0329% (w/v). It will be appreciated that any of the lower values for the concentration of α-Pinene mentioned herein may be combined with any of the upper values for the concentration of α-Pinene.

In a preferred embodiment, the plurality of terpenes comprises Limonene.

The concentration of Limonene in the cannabinoid-based formulation may be at least 0.0001 (w/v), or at least 0.001% (w/v). Preferably, the concentration of Limonene in the cannabinoid-based formulation may be at least 0.005% (w/v), at least 0.01% (w/v), or at least 0.02% (w/v). The concentration of Limonene in the cannabinoid-based formulation may be less than 0.75% (w/v), less than 0.5% (w/v) or less than 0.3% (w/v). The concentration of Limonene in the cannabinoid-based formulation may be less than 0.2% (w/v), less than 0.15% (w/v), or less than 0.1% (w/v). The concentration of Limonene in the cannabinoid-based formulation may be between 0.0001 and 0.5% (w/v), between 0.01 and 0.3% (w/v) or between 0.01 and 0.2% (w/v). The concentration of Limonene in the cannabinoid-based formulation may be between 0.01 and 0.05% (w/v), between 0.01 and 0.04% (w/v) or between 0.01 and 0.03% (w/v). The concentration of Limonene in the cannabinoid-based formulation may be between 0.03 and 0.09% (w/v), between 0.03 and 0.08% (w/v), or between 0.03 and 0.07% (w/v). Preferably, the concentration of Limonene in the cannabinoid-based formulation is about 0.02% (w/v). Preferably, the concentration of Limonene in the cannabinoid-based formulation is about 0.0225% (w/v). Preferably, the concentration of Limonene in the cannabinoid-based formulation is about 0.060%. More preferably, the concentration of Limonene in the cannabinoid-based formulation is about 0.0329%. It will be appreciated that any of the lower values for the concentration of Limonene mentioned herein may be combined with any of the upper values for the concentration of Limonene.

In a preferred embodiment, the plurality of terpenes comprises ocimene, more preferably β-Ocimene.

The concentration of β-Ocimene in the cannabinoid-based formulation may be at least 0.0001 (w/v), or at least 0.001% (w/v). Preferably, the concentration of β-Ocimene in the cannabinoid-based formulation may be at least 0.005% (w/v), at least 0.01% (w/v), at least 0.02% (w/v), or at least 0.03% (w/v). The concentration of β-Ocimene in the cannabinoid-based formulation may be less than 0.75% (w/v), less than 0.5% (w/v) or less than 0.3% (w/v). The concentration of β-Ocimene in the cannabinoid-based formulation may be less than 0.2% (w/v), less than 0.15% (w/v), or less than 0.1% (w/v). The concentration of β-Ocimene in the cannabinoid-based formulation may be between 0.0001 and 0.5% (w/v), between 0.01 and 0.3% (w/v) or between 0.01 and 0.2% (w/v). The concentration of β-Ocimene in the cannabinoid-based formulation may be between 0.01 and 0.07% (w/v), between 0.01 and 0.06% (w/v), between 0.01 and 0.05% (w/v), or between 0.01 and 0.04% (w/v). The concentration of β-Ocimene in the cannabinoid-based formulation may be between 0.001 and 0.05% (w/v), between 0.001 and 0.04% (w/v), between 0.001 and 0.02% (w/v), or between 0.001 and 0.01% (w/v). Preferably, the concentration of β-Ocimene in the cannabinoid-based formulation is about 0.03% (w/v). Preferably, the concentration of β-Ocimene in the cannabinoid-based formulation is about 0.0302% (w/v). More preferably, the concentration of β-Ocimene in the cannabinoid-based formulation is about 0.01% (w/v). More preferably, the concentration of β-Ocimene in the cannabinoid-based formulation is about 0.008% (w/v). It will be appreciated that any of the lower values for the concentration of β-Ocimene mentioned herein may be combined with any of the upper values for the concentration of β-Ocimene.

In a preferred embodiment, the plurality of terpenes comprises caryophyllene, preferably β-Caryophyllene.

The concentration of β-Caryophyllene in the cannabinoid-based formulation may be at least 0.0001 (w/v), or at least 0.001% (w/v). Preferably, the concentration of β-Caryophyllene in the cannabinoid-based formulation may be at least 0.005% (w/v), at least 0.01% (w/v), at least 0.02% (w/v), at least 0.03% (w/v), or at least 0.04% (w/v). The concentration of β-Caryophyllene in the cannabinoid-based formulation may be less than 0.75% (w/v), less than 0.5% (w/v) or less than 0.3% (w/v). The concentration of β-Caryophyllene in the cannabinoid-based formulation may be less than 0.2% (w/v), less than 0.15% (w/v), or less than 0.1% (w/v). The concentration of β-Caryophyllene in the cannabinoid-based formulation may be between 0.0001 and 0.5% (w/v), between 0.01 and 0.3% (w/v) or between 0.01 and 0.2% (w/v). The concentration of β-Caryophyllene in the cannabinoid-based formulation may be between 0.01 and 0.08% (w/v), between 0.01 and 0.07% (w/v), between 0.01 and 0.06% (w/v), or between 0.01 and 0.05% (w/v). The concentration of β-Caryophyllene in the cannabinoid-based formulation may be between 0.01 and 0.1% (w/v), between 0.01 and 0.09% (w/v), between 0.01 and 0.08% (w/v), or between 0.03 and 0.08% (w/v). Preferably, the concentration of β-Caryophyllene in the cannabinoid-based formulation is about 0.05% (w/v). Preferably, the concentration of β-Caryophyllene in the cannabinoid-based formulation is about 0.0459% (w/v). Preferably, the concentration of β-Caryophyllene in the cannabinoid-based formulation is about 0.07% (w/v). Preferably, the concentration of β-Caryophyllene in the cannabinoid-based formulation is about 0.071% (w/v). More preferably, the concentration of β-Caryophyllene in the cannabinoid-based formulation is about 0.0671% (w/v). It will be appreciated that any of the lower values for the concentration of β-Caryophyllene mentioned herein may be combined with any of the upper values for the concentration of β-Caryophyllene.

In a preferred embodiment, the plurality of terpenes comprises humulene, preferably Alpha-Humulene.

The concentration of Alpha-Humulene in the cannabinoid-based formulation may be at least 0.0001 (w/v), or at least 0.001% (w/v). Preferably, the concentration of Alpha-Humulene in the cannabinoid-based formulation may be at least 0.005% (w/v), at least 0.01% (w/v), or at least 0.02% (w/v). The concentration of Alpha-Humulene in the cannabinoid-based formulation may be less than 0.75% (w/v), less than 0.5% (w/v) or less than 0.3% (w/v). The concentration of Alpha-Humulene in the cannabinoid-based formulation may be less than 0.2% (w/v), less than 0.15% (w/v), or less than 0.1% (w/v). The concentration of Alpha-Humulene in the cannabinoid-based formulation may be between 0.0001 and 0.5% (w/v), between 0.01 and 0.3% (w/v) or between 0.01 and 0.2% (w/v). The concentration of Alpha-Humulene in the cannabinoid-based formulation may be between 0.001 and 0.04% (w/v), between 0.001 and 0.03% (w/v) or between 0.001 and 0.02% (w/v). The concentration of Alpha-Humulene in the cannabinoid-based formulation may be between 0.01 and 0.04% (w/v), between 0.01 and 0.03% (w/v) or between 0.01 and 0.02% (w/v). The concentration of Alpha-Humulene in the cannabinoid-based formulation may be between 0.005 and 0.04% (w/v), between 0.005 and 0.03% (w/v), or between 0.005 and 0.02% (w/v). Preferably, the concentration of Alpha-Humulene in the cannabinoid-based formulation is about 0.02% (w/v). Preferably, the concentration of Alpha-Humulene in the cannabinoid-based formulation is about 0.0151% (w/v). Preferably, the concentration of Alpha-Humulene in the cannabinoid-based formulation is about 0.01% (w/v). More preferably, the concentration of Alpha-Humulene in the cannabinoid-based formulation is about 0.008% (w/v). It will be appreciated that any of the lower values for the concentration of Alpha-Humulene mentioned herein may be combined with any of the upper values for the concentration of Alpha-Humulene.

In the first preferred embodiment, the plurality of terpenes comprises Myrcene, preferably β-Myrcene.

The concentration of β-Myrcene in the cannabinoid-based formulation may be at least 0.0001 (w/v), or at least 0.001% (w/v). Preferably, the concentration of β-Myrcene in the cannabinoid-based formulation may be at least 0.005% (w/v), at least 0.01% (w/v), at least 0.02% (w/v), at least 0.03% (w/v), at least 0.04% (w/v), or at least 0.05% (w/v). The concentration of β-Myrcene in the cannabinoid-based formulation may be less than 0.75% (w/v), less than 0.5% (w/v) or less than 0.3% (w/v). The concentration of β-Myrcene in the cannabinoid-based formulation may be less than 0.2% (w/v), less than 0.15% (w/v), or less than 0.1% (w/v). The concentration of β-Myrcene in the cannabinoid-based formulation may be between 0.0001 and 0.5% (w/v), between 0.01 and 0.3% (w/v) or between 0.01 and 0.2% (w/v). The concentration of β-Myrcene in the cannabinoid-based formulation may be between 0.01 and 0.1% (w/v), between 0.01 and 0.08% (w/v), between 0.01 and 0.07% (w/v), or between 0.01 and 0.06% (w/v). Preferably, the concentration of β-Myrcene in the cannabinoid-based formulation is about 0.05% (w/v). Preferably, the concentration of β-Myrcene in the cannabinoid-based formulation is about 0.061% (w/v). More preferably, the concentration of β-Myrcene in the cannabinoid-based formulation is about 0.0731% (w/v). It will be appreciated that any of the lower values for the concentration of β-Myrcene mentioned herein may be combined with any of the upper values for the concentration of β-Myrcene.

In the first preferred embodiment, the plurality of terpenes comprises Linalool.

The concentration of Linalool in the cannabinoid-based formulation may be at least 0.0001 (w/v), or at least 0.001% (w/v). Preferably, the concentration of Linalool in the cannabinoid-based formulation may be at least 0.005% (w/v), or at least 0.01% (w/v). The concentration of Linalool in the cannabinoid-based formulation may be less than 0.75% (w/v), less than 0.5% (w/v) or less than 0.3% (w/v). The concentration of Linalool in the cannabinoid-based formulation may be less than 0.2% (w/v), less than 0.15% (w/v), or less than 0.1% (w/v). The concentration of Linalool in the cannabinoid-based formulation may be between 0.0001 and 0.5% (w/v), between 0.01 and 0.3% (w/v) or between 0.01 and 0.2% (w/v). The concentration of Linalool in the cannabinoid-based formulation may be between 0.01 and 0.05% (w/v), between 0.01 and 0.04% (w/v), between 0.01 and 0.03% (w/v), or between 0.01 and 0.02% (w/v). Preferably, the concentration of Linalool in the cannabinoid-based formulation is about 0.01% (w/v). Preferably, the concentration of Linalool in the cannabinoid-based formulation is about 0.0125% (w/v). Preferably, the concentration of Linalool in the cannabinoid-based formulation is about 0.0120% (w/v). More preferably, the concentration of Linalool in the cannabinoid-based formulation is about 0.0183% (w/v). It will be appreciated that any of the lower values for the concentration of Linalool mentioned herein may be combined with any of the upper values for the concentration of Linalool.

Preferably, the method comprises combining the cannabinoid and plurality of terpenes with a pharmaceutically acceptable excipient or carrier. Accordingly, in a preferred embodiment, the cannabinoid-based formulation further comprises a pharmaceutically acceptable excipient or carrier.

Preferably, the method comprises contacting the cannabinoid with the pharmaceutically acceptable excipient or carrier at a temperature of between about 10° C. and 70° C., preferably between about 15° C. and 60° C., and more preferably between about 20° C. and 50° C., to form a mixture of the cannabinoid and excipient or carrier. Preferably, the mixture of cannabinoid and excipient or carrier is agitated.

Preferably, the mixture of cannabinoid and excipient or carrier is contacted with the plurality of terpenes at a temperature of between about 10° C. and 70° C., preferably between about 15° C. and 60° C., and more preferably between about 20° C. and 50° C., to create the formulation of the invention.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a liquid, and the pharmaceutical composition is in the form of a suspension in solution. Liquid vehicles are used in preparing solutions, suspensions, emulsions, syrups, elixirs and pressurized compositions. The cannabinoid and terpenes may be dissolved or suspended in a pharmaceutically acceptable liquid vehicle such as water, alcohol, ionic buffered solution, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil).

Pharmaceutically acceptable carriers, excipients, and diluents are relatively inert substances that facilitate administration or a pharmaceutically effective substance and can be supplied as a liquid solutions or suspensions, as emulsions, or as solid forms suitable for dissolution or suspension in liquid prior to use. For example, an excipient can give forms suitable for consistency, or act as a diluent. Suitable excipients include, but are not limited to stabilizing agents, wetting and emulsifying agents, salts for varying osmolarity, encapsulating agents, pH buffering substances, and buffers. Such excipients include any pharmaceutical agent suitable for direct delivery to the subject (for example sub-lingually) which may be administered without undue toxicity. Pharmaceutical acceptable excipients include, but are not limited to, sorbitol, any of the various TWEEN compounds, and liquids such as water, saline, glycerol and ethanol. Pharmaceutically acceptable salts can be included therein, for example, mineral acid salts such as hydrochlorides, hydrobromides, phosphates, sulphates and the like; and the salts of organic acids such as acetates, propionates, malonates, or benzoates.

In some embodiments, pharmaceutical acceptable excipients may include pharmaceutical acceptable carriers. Such pharmaceutically acceptable carriers can be sterile liquids, such as water and oil, including those of petroleum, animal, vegetable or synthetic origin, such as peanut oil, soybean oil, or mineral oil. Saline solutions and aqueous dextrose, polyethylene glycol (PEG) and glycerol solutions can also be employed as liquid carriers, particularly for sub-lingual solutions. Additional ingredients may also be used, for example preservatives, buffers, tonicity agents, antioxidants and stabilizers, non-ionic wetting or clarifying agents, or viscosity-increasing agents. A thorough discussion of pharmaceutical acceptable excipients and carriers is available in Remington's Pharmaceutical Sciences (Ed Remington J P and Gennaro A R; Mack Pub. Co. Easton, Pa 1990).

Most preferably, however, the pharmaceutically acceptable excipient or carrier comprises a medium-chain triglyceride (MCT), preferably MCT oil. Accordingly, in a preferred embodiment, the cannabinoid-based formulation further comprises a medium-chain triglyceride (MCT), preferably MCT oil. Suitable MCTs may be selected from a group consisting of a triglycerol linker with three fatty acid residues with carbon chain lengths ranging from 1 to 14. The chemical formula of each of these MCTs is shown below:

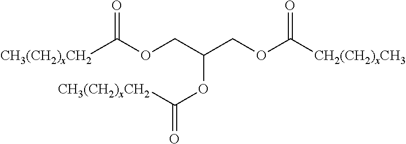

where X=1-14.

The MCT oil is preferably sourced from fractionated coconut oil. Preferred triglycerides comprise a carbon chain length or C6, C8, C10 or C12. Preferably, the triglyceride is almost all C8, which is highly refined.

The concentration of MCT oil in the cannabinoid-based formulation may be at least 80% (w/v), at least 82% (w/v) or at least 84% (w/v). Preferably, the concentration of MCT oil in the cannabinoid-based formulation may be at least 86% (w/v), or at least 88% (w/v). The concentration of MCT oil in the cannabinoid-based formulation may be less than 96% (w/v), less than 95% (w/v) or less than 94% (w/v). The concentration of MCT oil in the cannabinoid-based formulation may be less than 92% (w/v), or less than 90% (w/v). The concentration of MCT oil in the cannabinoid-based formulation may be between 85 and 93% (w/v), between 86 and 92% (w/v), between 87 and 91% (w/v) or between 88 and 90% (w/v). Preferably, the concentration of MCT oil in the cannabinoid-based formulation is about 89.8% (w/v). Preferably, the concentration of MCT oil in the cannabinoid-based formulation is about 89.8013% (w/v). More preferably, the concentration of MCT oil in the cannabinoid-based formulation is about 89.7597% (w/v). It will be appreciated that any of the lower values for the concentration of MCT oil mentioned herein may be combined with any of the upper values for the concentration of MCT oil.

The cannabinoid-based formulation of the invention is preferably in a liquid form. The formulation may comprise a tincture.

Preferably, the formulation is administrable by sublabial, buccal, sublingual, or oropharyngeal administration delivery. The preparation of the liquid formulations for sublabial, buccal, oropharyngeal or sublingual (oral) delivery allows the introduction of the active agents (i.e., the cannabinoid and the terpenes) into the systemic circulation via a mucous membrane, increasing the onset of activity and potency due to bypassing first-pass metabolism and increasing the drug bioavailability. Advantageously, given the lipophilicity and poor bioavailability of oral administration of cannabinoids for gastrointestinal absorption, which is considered the most popular method currently in the market for the delivery of medicines, the formulations of the invention surprisingly enable administration by this route. The liquid formulations may also be prepared for inhaled or nebulised delivery to the respiratory tract, as well as formulations for topical application to the skin and mucosal surfaces.

The formulation may be administered at least once a day, at least once a week, or at least once a month. The formulation may be administered two or three times a day, two or three times a week, or two or three times a month.

The formulation may be administered to a “subject” in need of the treatment, which may be a vertebrate, mammal, or domestic animal. Hence, the formulations according to the invention may be used to treat any mammal, for example livestock (e.g., a horse), domestic pets, or may be used in other veterinary applications. Most preferably, however, the subject is a human being.

A “therapeutically effective amount” of the formulation is any amount which, when administered to the subject, is the amount of the aforementioned that is needed to treat the target medical condition.

Preferably, the pain, inflammation, autoimmune disease, neurological disease, neoplasm, nausea, hypertension, hypertrophic cardiomyopathy, or fibrosis, are selected from the group of conditions consisting of: chemotherapy-induced peripheral neuropathy (CIPN), endometriosis, osteoarthritis, rheumatoid arthritis, ulcerative colitis, Crohn's disease, fibromyalgia, irritable bowel syndrome, asthma, chronic obstructive pulmonary disease (COPD), gout, scleroderma, lupus, Ehlers-Danlos syndrome, pericarditis, myocarditis, myocardial infarction, chemotherapy-induced nausea, anxiety, Type I diabetes, cardiovascular disease, primary hypertension, secondary hypertension, resistant hypertension, isolated systolic hypertension, malignant hypertension, obstructive hypertrophic cardiomyopathy, nonobstructive hypertrophic cardiomyopathy, pulmonary fibrosis, liver fibrosis, heart fibrosis, kidney fibrosis, mediastinal fibrosis, retroperitoneal cavity fibrosis, bone marrow fibrosis, skin fibrosis, and scleroderma.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

FIG. 1 is a flow diagram showing a first embodiment of a method for producing a CBD formulation of the invention comprising a CBD isolate, terpene isolates and an MCT oil, as excipient.

FIG. 2 is a flow diagram showing a second embodiment of a method for producing a CBD formulation of the invention comprising a CBD isolate, terpene isolates and an MCT oil, as excipient.

FIG. 3 shows a Certificate of Analysis (COA) for one embodiment of the CBD raw material.

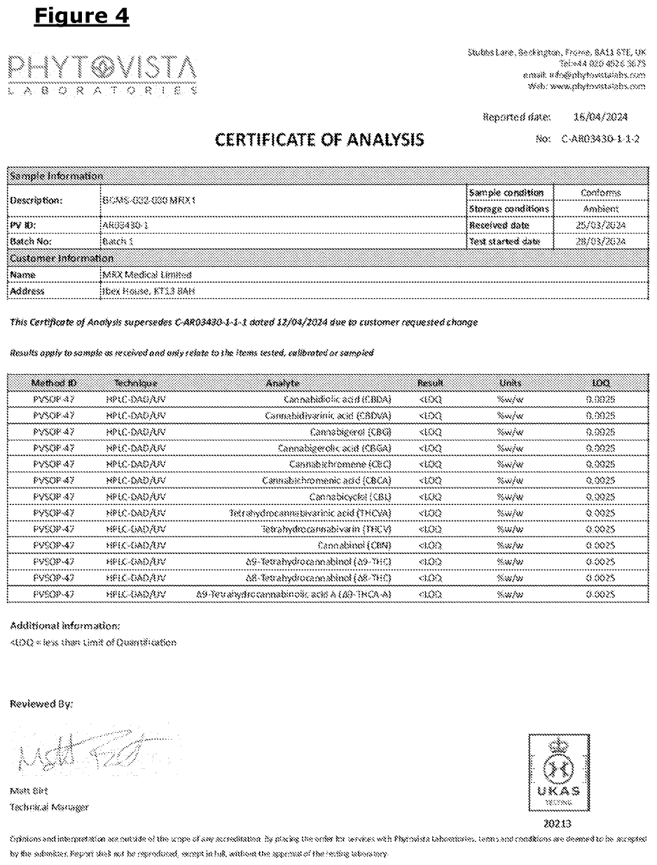

FIG. 4 shows a Certificate of Analysis (COA) that the cannabinoid-based formulation of the invention (referred to as MRX1) substantially lacks 13 non-CBD cannabinoid analytes.

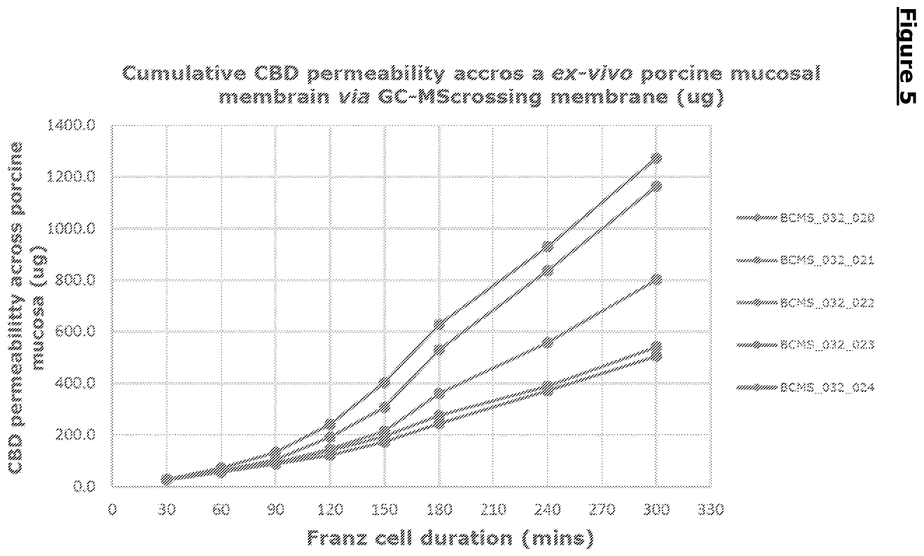

FIG. 5 shows the cumulative concentration of CBD permeation across porcine mucosal tissue membrane over time.

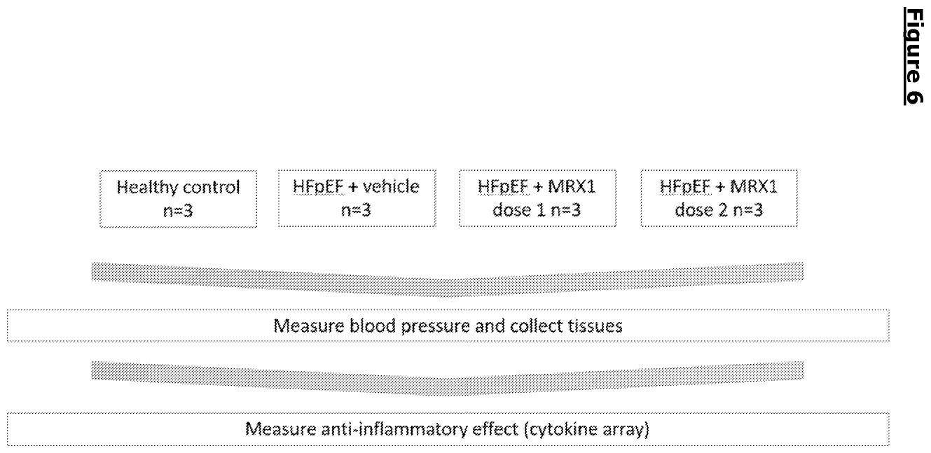

FIG. 6 shows the experimental design of the pilot study of the effect of the cannabinoid-based formulation of the invention (MRX1) in a murine model of heart failure with preserved ejection fraction (HFpEF).

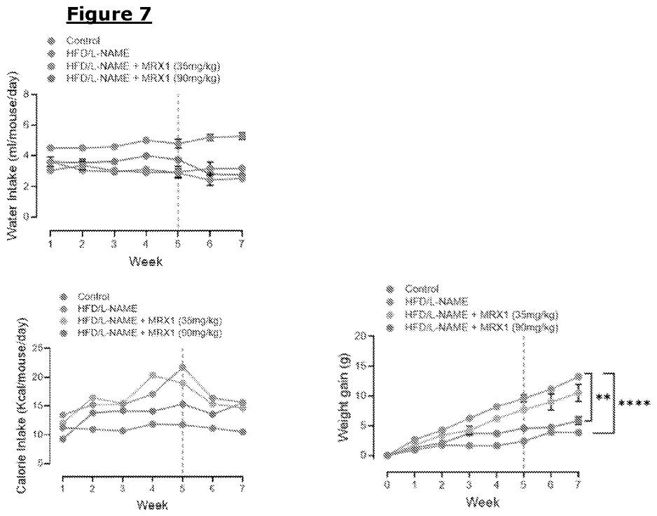

FIG. 7 shows mouse water and calorie intake and weight gain from baseline from the pilot study of the effect of the cannabinoid-based formulation of the invention in a murine model of heart failure with preserved ejection fraction (HFpEF).

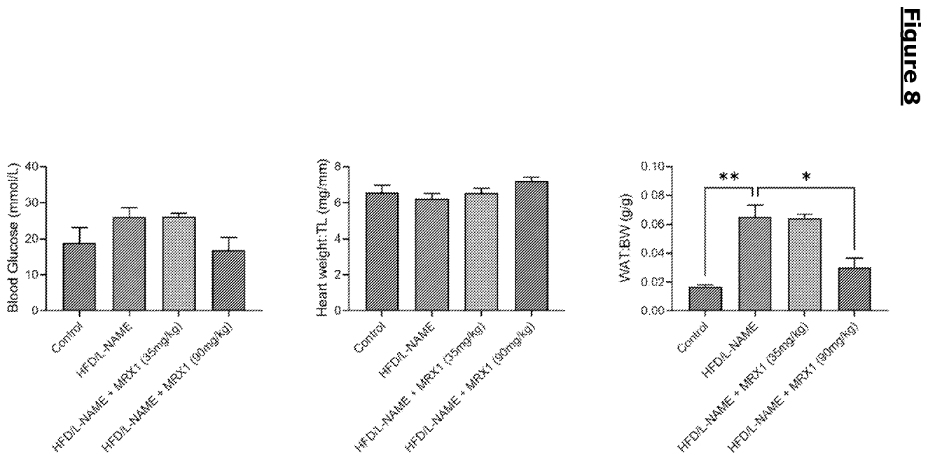

FIG. 8 shows changes in mouse blood glucose, heart weight, and white adipose tissue content (both corrected for body length/weight) from the pilot study of the effect of the cannabinoid-based formulation of the invention in a murine model of heart failure with preserved ejection fraction (HFpEF). *P<0.05, **P<0.01.

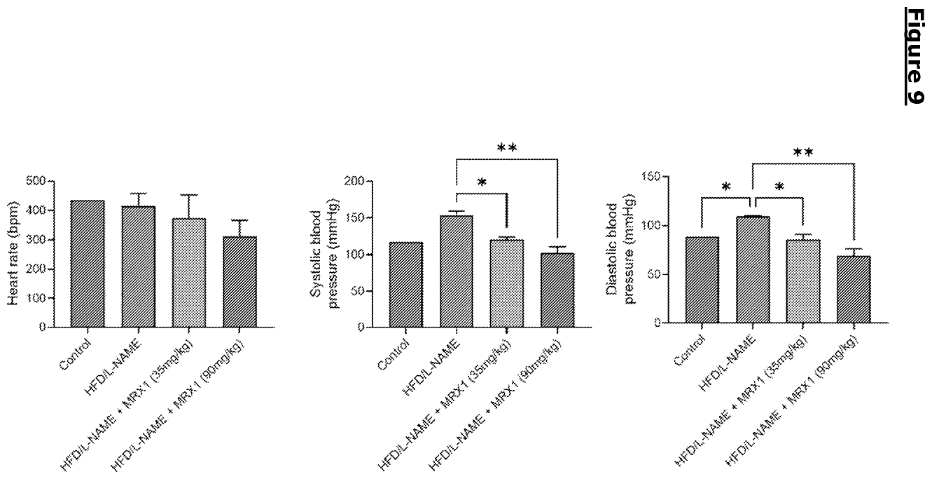

FIG. 9 shows effects of HFD/LNAME and the cannabinoid-based formulation of the invention on cardiovascular variables from the pilot study of the effect of the cannabinoid-based formulation of the invention in a murine model of heart failure with preserved ejection fraction (HFpEF). *P<0.05, **P<0.01.

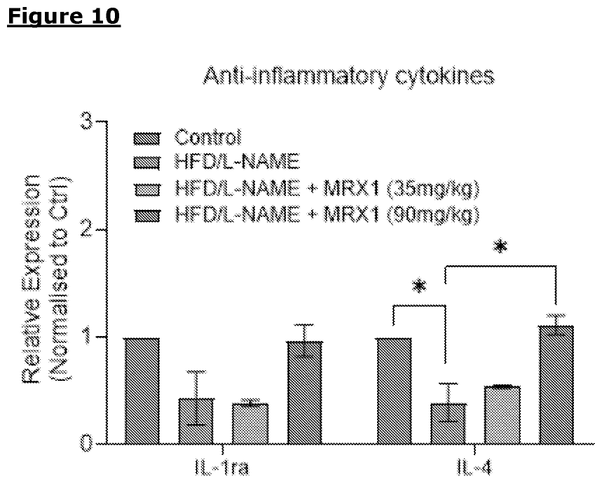

FIG. 10 shows protein expression of anti-inflammatory cytokines in mouse cardiac tissue from the pilot study of the effect of the cannabinoid-based formulation of the invention (MRX1) in a murine model of heart failure with preserved ejection fraction (HFpEF). *P<0.05.

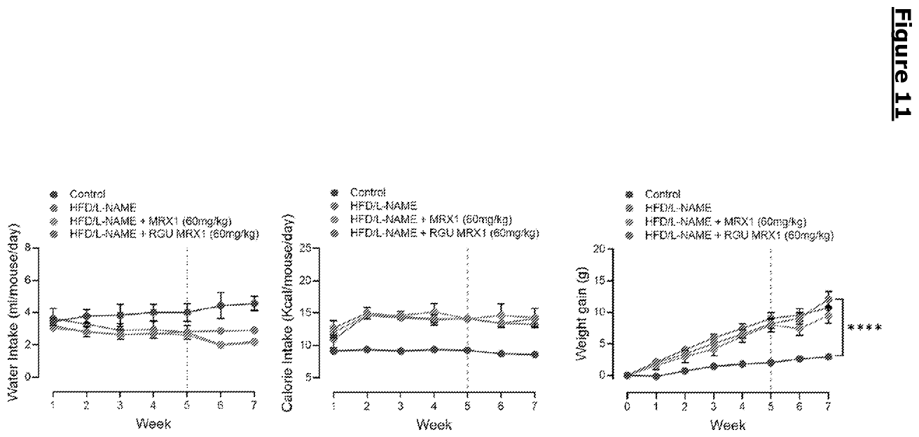

FIG. 11 shows mouse water and calorie intake, and weight gain from baseline, in normal fed and HFD/L-NAME mice with or without the cannabinoid-based formulation of the invention (MRX1) in a murine model of heart failure with preserved ejection fraction (HfpEF).

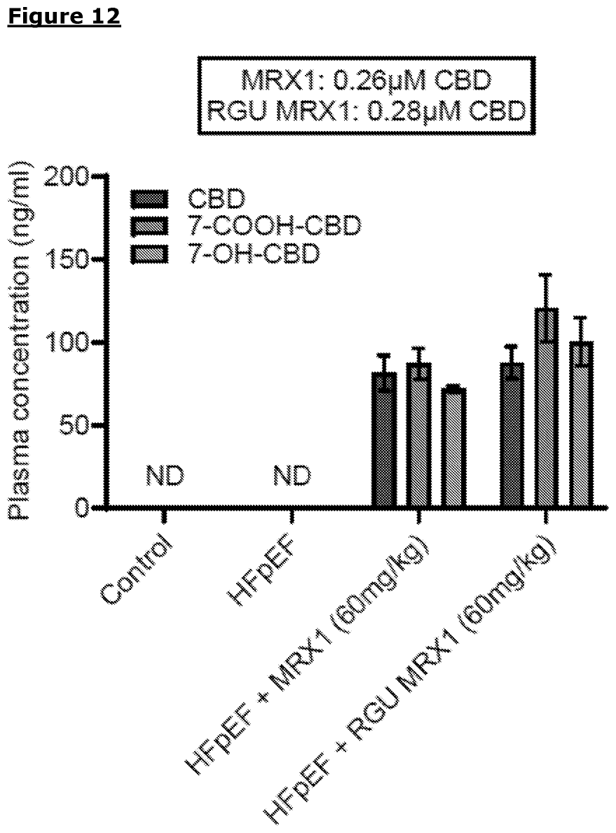

FIG. 12 shows mouse plasma levels of CBD and its metabolites in a murine model of heart failure with preserved ejection fraction (HfpEF).

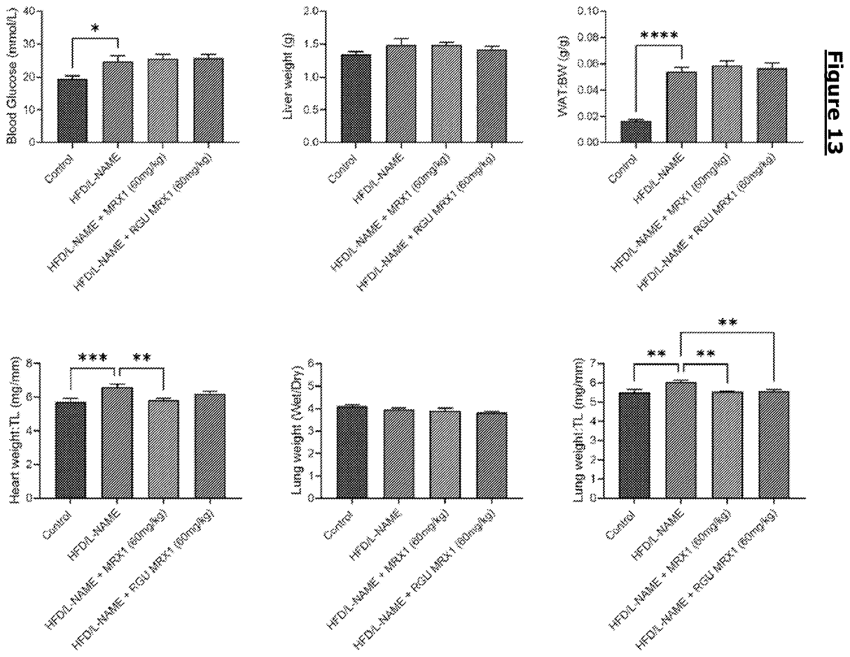

FIG. 13 shows mouse blood glucose levels and tissue weights in a murine model of heart failure with preserved ejection fraction (HfpEF). *P<0.05; **P<0.01.

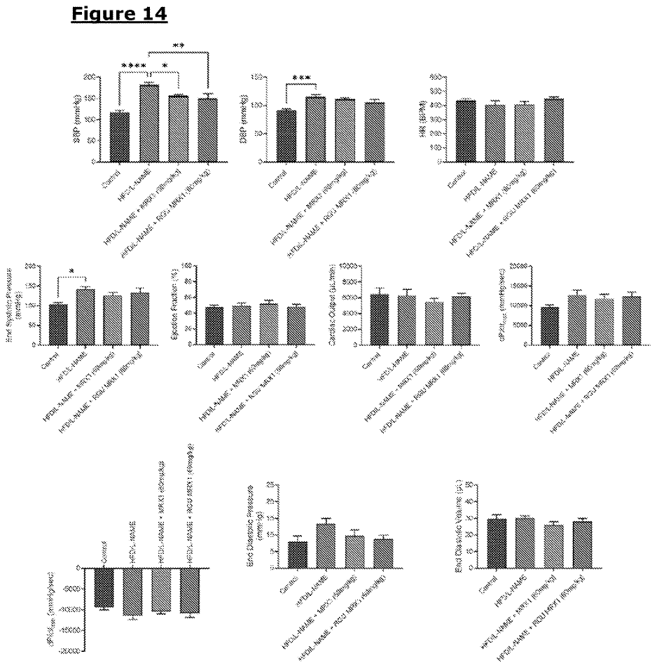

FIG. 14 shows changes in mouse blood pressure and heart rate (top panel), systolic function (middle panel), and diastolic function (bottom panel) in response to HFD/L-NAME with or without the cannabinoid-based formulation of the invention (MRX1) or RGU MRX1 in a murine model of heart failure with preserved ejection fraction (HfpEF). *P<0.05, **P<0.01, ***P<0.001.

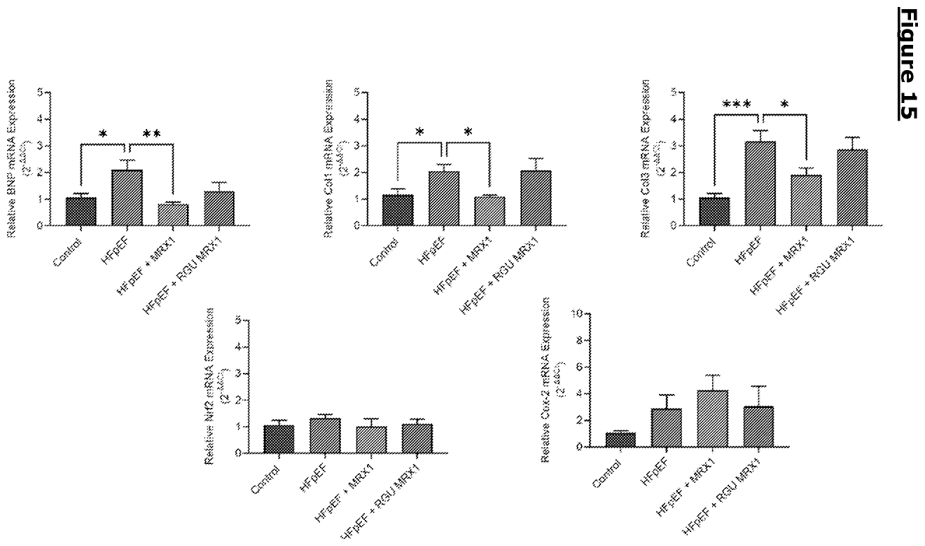

FIG. 15 shows expression of markers of heart failure, fibrosis, and inflammation in heart tissue from HFpeF mice treated with vehicle or the cannabinoid-based formulation of the invention (MRX1) in a murine model of heart failure with preserved ejection fraction (HfpEF). *P<0.05; **P<0.01; ***P<0.001.

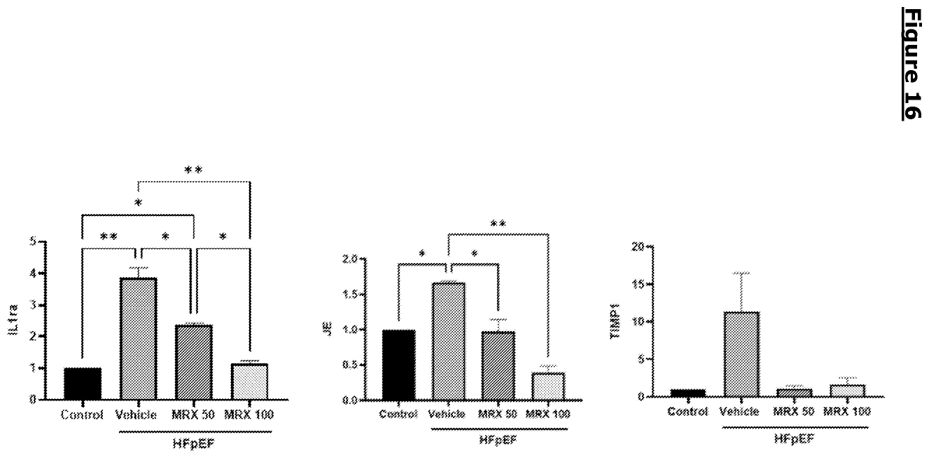

FIG. 16 shows expression of IL1ra, JE, and TIMP1 in adipose tissue from HfpeF mice treated with vehicle or the cannabinoid-based formulation of the invention (MRX1) in a murine model of heart failure with preserved ejection fraction (HfpEF). *P<0.05; **P<0.01; ***P<0.001.

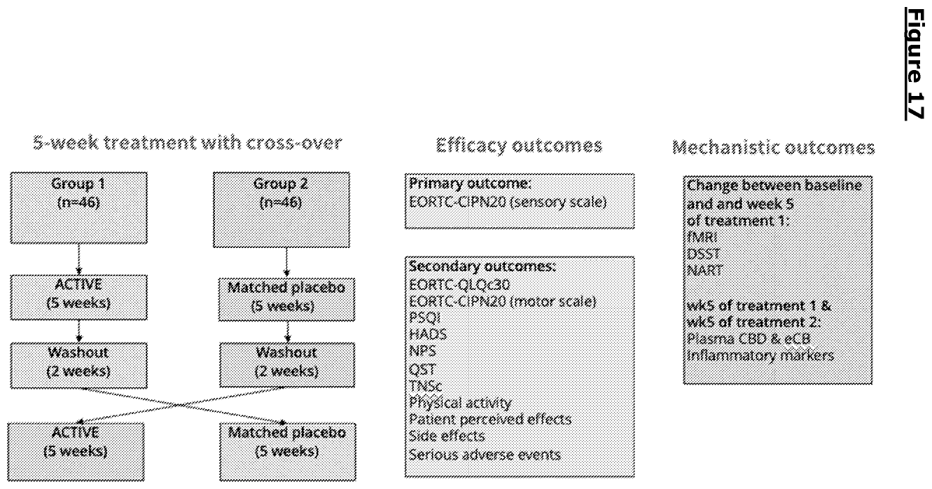

FIG. 17 shows the design a Phase II placebo controlled double-blind cross-over trial of the cannabinoid-based formulation of the invention for treating Chemotherapy Induced Peripheral Neuropathy (CIPN).

### EXAMPLES

### Background

The inventors have devised a novel method for reproducibly and consistently producing a highly standardised formulation comprising a cannabinoid (e.g., CBD) and a mixture of specific terpenes from scratch, for use in treating pain, inflammation, autoimmune disease, neurological disease, neoplasm, nausea, hypertension, hypertrophic cardiomyopathy, and fibrosis. The method involves combining an isolate of CBD of known purity, a highly defined mix of terpenes of known purity, and a medium chain triglycerides (MCT) oil of known purity, allowing the synthesis of a formulation with precisely standardised amounts of each component, whilst simultaneously minimising any contamination with THC.

### Example 1—Method of Producing the CBD Formulation

Referring to FIGS. 1 and 2, the inventors have designed a novel method for producing a highly uniform and reproducible CBD-based formulation (referred to herein as MRX1). The method involves the following steps:
1. Extracting CBD from a Cannabis sativa plant to produce a CBD extract;
2. Purifying a CBD isolate (at a defined purity) from the CBD extract;
3. Extracting terpenes from the appropriate species of plant to produce a terpene extract (i.e. an essential oil);
4. Purifying a terpene (at a defined purity) from the terpene extract, several of which are then blended together to create a terpene blend;
5. Preparing an MCT oil (at a defined purity);
6. Mixing together the CBD isolate, the terpene blend and the MCT oil to produce the CBD formulation of the invention.

Each of these steps will now be described in detail.

### Example 2—Preparing the CBD Isolate

The inventors have used two different embodiments of a method for extracting the pure CBD isolate, i.e., solvent extraction (shown in FIG. 1) and enzyme extraction (shown in FIG. 2).

### Solvent Extraction (FIG. 1)

Referring to FIG. 1 (left-hand side), in a first embodiment, the CBD isolate is prepared with the use of solvent extraction (e.g., using either an alcohol or an alkane) from suitable plant material (Cannabis sativa inflorescence and/or leaves), producing a slurry which is then filtered to produce a crude liquid extract. The crude extract is chilled to preferably −80° C. for 24 hours in a process called “Winterisation” in order to precipitate fats and waxes that are subsequently filtered off to produce a so-called ‘winterised’ extract.

The solvent (i.e., alcohol or alkane) is then removed under reduced pressure and the remaining crude oil ‘decarboxylated’ to convert the acidic compounds to neutral forms, and this is typically performed at 120° C. for at least 30 minutes, ideally at least one hour. The resulting decarboxylated extract is further purified by short path distillation at 130° C.-180° C. to remove residual extraneous non-cannabinoid materials. The resulting material is dissolved in alcohol (i.e., a solvent) to around the saturation point of the solution, before an alkane (acting as an anti-solvent) is slowly added to induce crystallisation. Upon completion of the first crystallisation (~95% purity CBD), the solid material is filtered off and re-dissolved in alcohol, and then a second crystallisation is performed to produce isolated CBD of the required specification.

### Enzyme Extraction (FIG. 2)

With reference to FIG. 2, in a second embodiment, the one or more cannabinoid is prepared by using enzyme digestion of plant material (e.g., inflorescence and/or leaves of Cannabis sativa). The plant material can comprise a moisture content of about 40%. The plant material is contacted with an enzyme solution which comprises a combination of one or more of the following enzymes: cellulase, beta-glucosidase, hemicellulase, xylanase, glucanase, beta-glucanase, pectinase, amylase, alpha-amylase, phospholipase, beta-mannanase, arabinanase, phytase and protease. The pH of the enzyme solution is adjusted to about pH5.6 using citric acid. A weight ratio of 1-10% (wt) of plant material is used to 99-10% (wt) enzyme solution. The plant material is macerated before, during and/or after contacting with the enzyme solution. In addition to the enzyme solution, the plant material is also contacted with a lipid for extracting the cannabinoid (i.e., CBD). For example, the lipid is vegetable oil, MCT oil, seed oil or olive oil. The weight ratio of lipid to plant material is about 1:1.5. The plant material is also macerated before, during and/or after contacting with the lipid.

The mixture of plant material, enzymes and lipid is then stirred and macerated at 10° C.-50° C. for about an hour. Upon full breakdown and dissolution of the plant material, the oil and water phases are then separated by gravity, centrifugation, filtration or a combination thereof. The oil layer is transferred to a separate vessel having agitation capability. The oil layer is contacted with an aqueous base, such as 0.1M NaOH or KOH, at a pH greater than or equal to 12. The weight of aqueous basic solution relative to the cannabinoid-containing oil is 1:5. This mixture is agitated for at least 20 minutes at a temperature of 40° C.-80° C.

On completion of the previous step, the mixture is contacted with a combination of: (i) sodium chloride and/or calcium carbonate; and (ii) glucose and/or fructose, and then agitated for a further 20 minutes. Upon completion, the layers are separated by gravity or centrifugation, and the aqueous layer may then be separated off. The pH of the separated aqueous layer is reduced to a pH of about 1 by contacting the aqueous layer with 85% ortho-phosphoric acid. The solution is agitated for a further 20 minutes, causing precipitation of cannabinoid acid salts. This liquid is filtered, giving a cannabinoid-rich solid precipitate between 65%-90% total cannabinoid content. The cannabinoid-rich precipitate is heated to at least 120° C. for 60 mins, or until such time that complete decarboxylation has been achieved. The resulting material is dissolved in a solvent (e.g., alcohol) to around the saturation point of the solution, before an anti-solvent (e.g., alkane) is (slowly) added to induce crystallisation. Optionally, the solid material can be filtered off and re-dissolved in solvent (e.g., alcohol), and a second crystallisation may be performed to produce an isolated cannabinoid (preferably, CBD) of the required specification.

### Certificate of Analysis (COA)

Referring to FIG. 3, there is shown a Certificate of Analysis (COA) for one embodiment of the CBD raw material. As can be seen, a wide number of parameters have been measured, and are well within accepted limits. In particular, the inventors note the ability to set the specification for d9-tetrahydrocannabinol (THC) to 0.02%, which is significantly lower than the pharmaceutical requirements of less than, or equal to, 0.15% for any single known organic impurity.

### Example 3—Preparing the Terpene Blend

Referring to FIGS. 1 and 2 (right-hand side), the terpenes are prepared by_steam distillation to produce an essential oil from the appropriate species of plant, such as lavender. The resulting essential oil is purified to individual components by flash chromatography with silica. The following example is for the purification of linalool from lavender, though it will be appreciated that other essential oils comprising one or more terpene can be prepared from other plant species.

The steam distillation equipment is a closed system consisting of a boiling vessel into which macerated lavender is placed, adequate water is then added to allow for mobilisation of the plant material. Steam is introduced into the bottom of the vessel via a dip tube running from the top of the boiling vessel. The mixture is heated to boiling point and the vapour travels through a manifold at the top of the boiling vessel to a water-cooled condenser. The condensate is collected and allowed to separate, where the top essential oil layer is collected.

The essential oil is dissolved at the appropriate concentration in 90:10 heptane:ethyl acetate and introduced to a flash chromatography system and separated using a silica packed column and 90:10 heptane:ethyl acetate mobile phase. The later fractions yield>95% purity linalool.

The inventors used isolated terpenes after the purification process as raw materials for preparation of the CBD formulation. The purity standard of the raw terpenes is 95% purity and above (measured by GC). Impurities are mostly other terpenes, and all impurities above 0.2% are identified in order to be included as part of the formulation and to ensure the highest consistency.

The terpenes are then combined by weight to produce a highly reproducible terpene mixture or blend. As shown in FIGS. 1 and 2, the resulting essential oils, each comprising a terpene at a 95% purity or above, are mixed together to form a terpene blend, i.e., terpene X, terpene Y and/or terpene Z, and so on), each at a purity of 95% or more. Table 2 shows one exemplary embodiment of the terpene blend.

TABLE 2
Terpene blend

| Ingredient | % |
|---|---|
| Beta-Myrcene | 14.29 |
| Beta-Caryophyllene | 14.29 |
| Beta-Ocimene | 14.29 |
| Alpha-Pinene | 14.29 |
| Limonene | 14.29 |
| Alpha-Humulene | 14.29 |
| Linalool | 14.29 |
| Total | 100.00 |

### Example 4—Preparing the MCT Oil

Referring to FIGS. 1 and 2 (left-hand side), a commercially available pharmaceutical grade MCT oil is obtained from H Plus Limited, Walker House, Exchange Flags, Liverpool, L2 3YL, England. For example, the MCT can be sourced from fractionated coconut oil, and has a carbon chain length of C6, C8, C10 or C12, though a highly refined C8 chain length is preferred.

### Example 5—CBD Formulation

The method comprises combining the CBD isolate from Example 2, the terpene isolate from Example 3, and the MCT oil from Example 4 to produce the cannabinoid-based formulation of the invention. This is achieved by first mixing the CBD isolate with the MCT oil with agitation at about 20° C.-50° C. Then, the CBD/MCT solution is mixed with the plurality of blended terpenes with agitation at about 20° C.-50° C. to create the CBD/MCT/terpene formulation.

The inventors have designed an embodiment of the CBD-based formulation with the composition shown in Table 3.

TABLE 3
A first embodiment of a CBD Formulation

| | % Content | % Low | % High |
|---|---|---|---|
| Cannabinoid | | | |
| CBD | 10 | 8 | 12 |
| Terpenes | | | |
| Beta-myrcene | 0.0500 | 0.01 | 0.2 |
| Beta-caryophyllene | 0.0459 | 0.01 | 0.2 |
| Beta-Ocimene | 0.0302 | 0.01 | 0.2 |
| Alpha-pinene | 0.0225 | 0.01 | 0.2 |
| limonene | 0.0225 | 0.01 | 0.2 |
| Alpha-humulene | 0.0151 | 0.01 | 0.2 |
| Linalool | 0.0125 | 0.01 | 0.2 |
| MCT Oil | 89.8013 | | |
| Total | 100 | | |

The inventors have designed another embodiment of the CBD-based formulation with the composition shown in Table 7.

TABLE 7
A second embodiment of a CBD Formulation

| | % Content | % Low | % High |
|---|---|---|---|
| Cannabinoid | | | |
| CBD | 10 | 8 | 12 |
| Terpenes | | | |
| Beta-myrcene | 0.0610 | 0.01 | 0.2 |
| Beta-caryophyllene | 0.0710 | 0.01 | 0.2 |
| Beta-Ocimene | 0.008 | 0.01 | 0.2 |
| Alpha-pinene | 0.0230 | 0.01 | 0.2 |
| limonene | 0.060 | 0.01 | 0.2 |
| Alpha-humulene | 0.008 | 0.01 | 0.2 |
| Linalool | 0.0120 | 0.01 | 0.2 |
| MCT Oil | 89.8013 | | |
| Total | 100 | | |

The inventors have designed another, and preferred, embodiment of the CBD-based formulation with the composition shown in Table 8.

TABLE 8
A third embodiment of a CBD Formulation

| | % Content | % Low | % High |
|---|---|---|---|
| Cannabinoid | | | |
| CBD | 10 | 8 | 12 |
| Terpenes | | | |
| Beta-myrcene | 0.0731 | 0.01 | 0.2 |
| Beta-caryophyllene | 0.0671 | 0.01 | 0.2 |
| Beta-Ocimene | 0.0080 | 0.01 | 0.2 |
| Alpha-pinene | 0.0329 | 0.01 | 0.2 |
| limonene | 0.0329 | 0.01 | 0.2 |
| Alpha-humulene | 0.0080 | 0.01 | 0.2 |
| Linalool | 0.01830 | 0.01 | 0.2 |
| MCT Oil | 89.7597 | | |
| Total | 100 | | |

### Example 6—CBD-Only Formulation (MRX1)

Analysis of the cannabinoid-based formulation of the invention, referred to herein as MRX1, was performed by Phytovista Laboratories, a specialist ISO accredited laboratory which test CBD & hemp products. Using HPLC-DAD and UV-spectrophotometry, Phytovista Laboratories tested the MRX1 for the presence of 13 non-CBD cannabinoid analytes, namely Cannabidiolic acid (CBDA), Cannabidivarinic acid (CBDVA), Cannabigerol (CBG), Cannabigerolic acid (CBGA), Cannabichromene (CBC), Cannabichromenic acid (CBCA), Cannabicyclol (CBL), Tetrahydrocannabivarinic acid (THCVA), Tetrahydrocannabivarin (THCV), Cannabinol (CBN), Δ9-Tetrahydrocannabinol (Δ9-THC), Δ8-Tetrahydrocannabinol (Δ8-THC), Δ9-Tetrahydrocannabinolic acid A (Δ9-THCA-A).

As shown in FIG. 4, Phytovista Laboratories issued a certificate of analysis confirming that MRX1 contained less than the limit of quantification (<LOQ), where the LOQ was 0.0025, for all 13 analytes.

### Example 7—MRX1 Bioavailability

A mucosal skin permeation study of the cannabinoid-based formulation of the invention, referred to herein as MRX1, via Franze cell analysis was performed by Nottingham Trent University.

### Aim

The aim of the study was to determine the systemic exposure, over time, of MRX1 in a porcine (pig) mucosal tissue permeation study, using an in vitro model (Franz cell apparatus) utilising a proprietary transdermal formulation, i.e., an embodiment of the cannabinoid-based formulation of the invention, containing cannabinoid isolate cannabidiol (CBD) in a lipid matrix (comprising of combinations of the following: medium chain triglycerides (MCT), propylene glycol, ethanol and terpenes (β-Myrcene, β-Caryophyllene, β-Ocimene, α-Pinene, Limonene, α-Humulene, and linalool).

### Materials and Instrumentation

MRX1, Cannabidiol (CBD) isolate, Kollisolv MCT70, propylene glycol, and anhydrous ethanol were supplied to the University. The cannabidiol reference standard was used as received by the supplier (Restek, #34011). All other solvents were used as supplied (Analytical or LCMS grade; Fisher Scientific and Sigma) without further purification. Apparatus used included DixonScience Franz cell apparatus (#XFCDR01), and Agilent 7890A series Gas Chromatography coupled with Agilent 5975C MDS with ALS 7693 supported with NIST library search.

### Method

### Franz Cell Protocol

Franz cell experiments were carried out on the single MRX1 formulation (0.5 mL sample containing CBD 10-20% w/v) in triplicate for five sperate formulations, namely BCMS_032_020, BCMS_032_021, BCMS_032_022, BCMS_032_023, and BCMS_032_024. Each Franz cell was carried out over a five-hour period with specific sampling time points at time=0, 30, 60, 90, 120, 150, 180, 240 and 300 minutes using porcine mucosa tissue membranes.

In summary, the following procedure was carried out:

The porcine mucosa tissue membrane tissue was harvested from fresh (<4 hours) euthanised pigs (ca. 6 month old) before starting the procedure, ensuring to trim any excess tissue from the skin sample. All samples were washed and dried before loading into the Franz cell apparatus. Franz cell apparatus with a receptor fill volume of ca. 2.0 mL and an effective diffusional area of 1.33 cm² were used.

The receptor compartment of the Franz cell was filled (ca. 2.0 mL) with degassed phosphate-buffered saline solution (PBS—sodium chloride 8.0 g/L, potassium chloride 0.2 g/L, disodium hydrogen phosphate 1.15 g/L, potassium dihydrogen phosphate 0.2 g/L) with bovine serum albumin (4% v/v) at pH 7.4 (±0.1), maintained at 37° C. (±1° C.).

The prepared tissue was placed into the Franz cell apparatus and clamped into position, ensuring a firm seal. The skin was saturated in situ by placing a small amount (ca. 0.5 mL) of buffer medium on top on the skin/donor chamber for prior starting the analysis (1 hr). After ensuring the sample was saturated and there were no leaks in the system, buffer medium was removed from the donor chamber.

The Franz cell apparatus was continually checked to ensure there were no air bubbles present under the skin membrane; if present the sample chamber was carefully angled to remove them. The temperature of the system was maintained at 37° C. (±1° C.) with 5% CO₂ and kept under constant stirring using a magnetic stirrer bar to ensure homogeneous mixing of the receptor solution throughout the analysis.

The five suppled formulations for testing were individually applied (0.5 mL, stored at room temperature) to the receptor reservoirs for direct contact with the surface of the tissue membrane.

A pre-weighed sterilised Pasteur pipette was used to gently spread the sample onto the surface of the skin ensuring it covered the entire surface. The pipette was then reweighed to obtain the weight of any residue removed from the Franz cell apparatus. The timer was then started from 0 min. At each specified time-point (0, 30, 60, 90, 120, 150, 180, 240, and 300 min), all of the buffer phase (2 mL) was removed from the receptor chamber via a sterile syringe and needle. The sample aliquot was then flash frozen (with liquid nitrogen), and placed in the freezer (−20° C.) ready for chromatographic analysis. The receptor chamber was immediately replenished with fresh buffer solution (2 mL, equilibrated to 37° C.), ensuring complete filling of the chamber with no air bubbles.

After the final sampling time point (five hours), the stirrer was stopped and the Franz cell apparatus was carefully dismantled. The removed skin tissue sample was placed into a clean dry beaker and the donor chamber into a separate beaker.

Buffer solution (10 mL) was added to each beaker to thoroughly rinse the surfaces of the skin and donor chamber. The washings were then each flash frozen with liquid nitrogen and placed in the freezer (−20° C.) ready for chromatographic analysis.

The frozen skin samples were freeze dried, milled to fine powder, and weighed ready for chromatographic analysis.

### GC/MS Protocol

Agilent 7890A Gas Chromatography coupled with Agilent 5975C MDS mass spectrometer and GC column DB-5 ms (30.0 m×250 μm×0.25 μm nominal) Ultra inert size with constant follow of Helium gas (1 mL/min) in split mode (10:1) was utilised for all experiments.

All time point aliquot samples were allowed to come to room temperature and the CDB extracted with chloroform (2.0 mL) and dried with magnesium sulphate (ca. 0.2 g). The aqueous layer was discarded using an aspiration station (Gilson, UK) and the organic layer was transferred (1 mL) to amber GC/MS sample vials ready for analysis.

The samples (10 mL) collected from the washed Franz cell apparatus were extracted into chloroform (4.0 mL) and dried with magnesium sulphate (ca. 0.4 g). The aqueous layer was discarded using an aspiration station (Gilson, UK) and the organic layer was transferred (1 mL) to amber GC/MS sample vials ready for analysis.

Samples (10 mL) collected from the washed skin were extracted into chloroform (2.0 mL), dried with magnesium sulphate (ca. 0.2 g) and filtered before being transferred (1 mL) to amber GC/MS sample vials ready for analysis.

The powdered freeze dried skin samples were dispersed into chloroform (3×1.0 mL), filtered, dried with magnesium sulphate (ca. 0.2 g), and transferred (1 mL) to amber GC/MS sample vials ready for analysis.

The samples were loaded into the autosampler of the GM/MS. The GM/MS instrument was programmed to heat (300° C. with ramp 10° C./min) from initial temperature (130° C.) and held for 2 mins. The total run time per sample was 24 mins. The mass spectrometer was operated in full scan mode with scanning range of 40-450 amu and an ionisation energy of 70 eV.

### Results

Franz cell analysis studies of CDB permeability across porcine mucosal tissue

### 1. CBD Permeation Study Over Time

TABLE 4
The total of CBD permeation across the porcine
mucosal tissue membrane after 5 hours from formulations
BCMS_032_020, BCMS_032_021, BCMS_032_022, BCMS_{—}
032_023, and BCMS_032_024.

| | | | | After | |
|---|---|---|---|---|---|
| | CBD | Terpenes | MCT | total 5 | |
| Sample | w/v | w/v | w/v | hours | Relative |
| refence | (%) | (%) | (%) | (ug) | % |
| BCMS032020 | 10.00 | 0.250 | 100 | 1273 | 100.0 |
| BCMS032021 | 10.00 | 0.125 | 100 | 1163 | 91.4 |
| BCMS032022 | 10.00 | | 100 | 802 | 63.1 |
| BCMS032023 | 10.00 | | 50_80 | 505 | 39.7 |
| | | | (EtOH:PG) | | |
| BCMS032024 | 10.00 | 0.250 | 50_50 | 541 | 42.5 |
| | | | (EtOH:PG) | | |

(EtOH—ethanol, PG—propylene glycol, MCT—medium chain triglycerides.)

TABLE 5
The accumulative concentration of CBD permeation
across the porcine mucosal tissue membrane over time from formulations
BCMS_032_020, BCMS_032_021, BCMS_032_022, BCMS_{—}
032_023, and BCMS_032_024.

| | | BCMS_{—} | BCMS_{—} | BCMS_{—} | BCMS_{—} |
|---|---|---|---|---|---|
| | BCMS_{—} | 032_{—} | 032_{—} | 032_{—} | 032_{—} |
| Time | 032_{—} | 021 | 022 | 023 | 024 |
| (min) | 020(ug) | (ug) | (ug) | (ug) | (ug) |
| 30 | 28.8 | 27.5 | 26.5 | 27.3 | 26.7 |
| 60 | 71.8 | 61.3 | 55.7 | 55.9 | 55.9 |
| 90 | 132.0 | 103.6 | 91.9 | 87.0 | 90.8 |
| 120 | 241.5 | 191.6 | 145.8 | 121.0 | 139.3 |
| 150 | 402.2 | 306.1 | 214.8 | 172.9 | 193.8 |
| 180 | 626.9 | 529.6 | 360.0 | 244.1 | 275.4 |
| 240 | 930.1 | 837.1 | 557.1 | 371.3 | 388.4 |
| 300 | 1272.6 | 1163.5 | 802.4 | 505.1 | 541.0 |

Referring to FIG. 5, Franz cell analysis of CBD permeation studies across ex vivo porcine mucosa tissue membrane demonstrates that one embodiment of the cannabinoid-based formulation of the invention, sample BCMS_032_020, which contains 10% CBD, 0.250% terpenes, and 100% MCT, shows a higher percentage of CBD penetration across the porcine mucosal membrane, followed by another embodiment of the cannabinoid-based formulation of the invention, sample BCMS_032_021, which contains 10% CBD, 0.250% terpenes and 100% MCT. This confirms that the presence of terpenes increases the rate of CBD penetration across the skin membrane. The CBD permeation from the formulations based on Ethanol:Propylene glycol (50:50), namely samples BCMS_032_022 and BCMS_032_023, are significantly lower compared to the MCT formulations. Moreover, the sample BCMS_032_024 which consists of EtOH:Propylene glycol (50:50) along with terpenes, also demonstrated a reduction in CBD penetration compared to the MCT formulations.

### Example 8—MRX1 Formulation Substantially Lacks THC

Analysis of an embodiment of the cannabinoid-based formulation of the invention was performed by BCM Analytical Services, who are the contract testing business of the Fareva group, and provide full spectrum pharmaceutical analysis and testing. Using HPLC, BCM Analytical Services tested an embodiment of the cannabinoid-based formulation of the invention for impurities including d9-THC and d8-THC.

As shown in Table 6 below, the results of HPLC analysis of the formulation showed that both d9-THC and d8-THC were below the limit of detection (<LOD), thus confirming that the formulation of the invention substantially lacks THC.

TABLE 6
HPLC impurity profile analysis of an embodiment of
the cannabinoid-based formulation of the invention.

| | Impurities | | | | |
|---|---|---|---|---|---|
| Storage | d9-THC | d8-THC | CBN | Unidentified | Total |
| conditions | content^{a} | content^{a} | content^{a} | impurities | impurities |
| Initial | <LOD | <LOD | <LOD | 0.4^{b} | 0.4 |

LOD: Limit of detection
LOQ, Limit of Quantitation
^{a}LOD = 0.01%, LOQ = 0.02%
^{b}GB1-LABO-7744

### Example 9—Anti-Inflammatory Effects of MRX1

A study on the effect of an embodiment of the cannabinoid-based formulation of the invention, referred to herein as MRX1, in a murine model of heart failure with preserved ejection fraction (HfpEF) was performed by Dr Nadine Godsman and Dr Sarah Walsh at the Centre for Cardiometabolic Health Research and Robert Gordon University, Aberdeen, UK.

### Background

Heart failure with preserved ejection fraction (HfpEF) is a form of heart failure in which the ejection fraction (the percentage of the volume of blood within the left ventricle ejected during systolic contraction) is normal, but there is abnormal diastolic function resulting from increased stiffness of the left ventricle. This causes a decrease in left ventricular relaxation during diastole, with a resultant increased pressure and/or impaired filling. HfpEF is characterised by thickening of the ventricular wall (concentric hypertrophy), which leads to increased left ventricular mass and is typically accompanied by a normal, or slightly reduced, end diastolic filling volume.

Left ventricular hypertrophy in HfpEF occurs as a result of changes in the extracellular matrix surrounding the cardiomyocytes, whereby the process of fibrosis results in the laying down of additional collagen material, which contributes to the wall stiffening. The activation of the fibrotic process is multi-factorial, however activation of an inflammatory response, which attracts collagen-producing fibroblasts to the cardiac tissue, plays a major role. Although diverse factors contribute to the development of HfpEF, hypertension, obesity, metabolic syndrome, and sedentary lifestyle have been identified as important risk factors.

There is substantial evidence that cannabidiol (CBD) can act both as an anti-inflammatory agent and to protect the heart from damage against an ischaemia/reperfusion insult. The purpose of the study was to determine the ability of MRX1, an oil-based CBD preparation containing 10% CBD, to slow or prevent the development of the changes in cardiac structure and function in a pre-clinical murine model of HfpEF; this model emulates the risk factors of both hypertension and obesity through administration of the nitric oxide (NO) synthase inhibitor L-NAME and high fat feeding, respectively.

### Pilot Dose Selection Study

In order to ensure that a sufficiently high dose of MRX1 was to be used for a drug intervention study, a preliminary dosing study was undertaken. Male C57BL/6J mice (8-10 weeks old) were fed either a high fat diet with L-NAME (0.5 g/L) in drinking water or a matched control diet for 7 weeks. In the HFD/L-NAME mice, vehicle, MRX1 (35 mg/kg or 90 mg/kg, equivalent to 3.5 and 9 mg/kg CBD, respectively) were administered in the drinking water during weeks 6 & 7 of the diet intervention period. Cremophor was employed to achieve mixing of the MRX1 oil and the water. Animals were allowed free access to food and water. Daily measurement of water intake was performed to calculate the actual CBD intake in mg/kg, along with determination of food intake.

At the end of the intervention period, the mice were anaesthetised prior to blood pressure measurement via a carotid cannula. Following euthanasia by anaesthetic overdose, tissue and blood samples were taken for assessment of a range of variables. The experimental design of the pilot study is shown in FIG. 6.

### Results

Referring to FIG. 7, in response to the HFD/L-NAME intervention, water intake was unchanged, while calorie intake and weight gain increased compared to mice on a normal diet. Intervention with MRX1 did not alter either water or food intake, however the higher dose of MRX1 slowed down weight gain compared to vehicle.

Referring to FIG. 8, consumption of the HFD/L-NAME diet in mice given vehicle did not alter blood glucose levels or heart weight (corrected for tibial length), but did significantly increase white adipose tissue (WAT) to body weight ratio. Neither dose of MRX1 had any effect on either blood glucose levels or on HW:TL ratio. However, the increase in WAT:BW ratio was prevented in mice supplemented with the higher, but not the lower, dose of MRX1.

Referring to FIG. 9, in vehicle treated mice, HFD/LNAME diet caused an increase in both systolic and diastolic blood pressure compared to mice fed a normal diet, indicating the mice were hypertensive. MRX1 prevented this diet-induced rise in both systolic and diastolic blood pressure in a dose-dependent manner. Heart rate was unaffected by the diet alone or with the lower dose of MRX1. The higher dose of MRX1 appeared to reduced heart rate slightly, but this was not statistically significant.

To determine whether MRX1 had any effects on expression of inflammatory markers, a cytokine array was performed on samples of cardiac tissue. Referring to FIG. 10, HFD/LNAME treatment resulted in a suppression of two anti-inflammatory cytokines (IL-1ra and IL-4) in the heart, which was not present in mice given the higher dose of MRX1.

### Summary of Dose Selection Study

Although white adipose tissue (WAT) accumulation was reduced in mice treated with the higher concentration of MRX1 (90 mg/kg), the calorie intake was already reduced in those mice before MRX1 administration, so this effect is unlikely to be linked to treatment with MRX1. Both doses of MRX1 (35 mg/kg and 90 mg/kg) exerted anti-hypertensive effects, reducing both systolic and diastolic blood pressure in mice. The higher dose of MRX1 (90 mg/kg) also restored the reduced protein expression of anti-inflammatory cytokines in the heart seen with the diet. On this basis, a target dose of 100 mg/kg of MRX1 was chosen as the dose for the intervention study.

### Intervention Study to Determine the Effects of MRX1 in HfpEF

Having identified from the dose selection study that a dose of 9 mg/kg of MRX1 was both anti-hypertensive and raised anti-inflammatory cytokine levels in the heart, the purpose of the intervention study was to determine whether MRX1 could ameliorate both the functional and structural cardiac changes seen in HfpEF. Moreover, since certain terpenes have been shown to have independent cardioprotective properties, it was explored if raising the terpene levels in the MRX1 formulation preparation could provide added benefits.

As with the dose selection study, male C57BL/6J mice (8-10 weeks old, 12 mice per experimental group) were fed either a high fat diet with L-NAME (0.5 g/L) in drinking water or a matched control diet for 7 weeks. In the HFD/L-NAME mice, either vehicle, MRX1 (1 mg/ml in drinking water with a target intake of 100 mg/kg), or MRX1 with additional terpenes (extra 0.05% BCP, 0.01% Beta-pinene, and 0.001% farnesol; termed RGU MRX1) were administered in drinking water in weeks 6 & 7 of the dietary intervention period. Water intake was measured daily to calculate actual intake of the formulation.

### Results

Referring to FIG. 11, water intake in the normal fed mice and in vehicle treated HFD/L-NAME mice remained constant throughout the diet intervention period. However, both the MRX1 and the RGU MRX1 groups exhibited a slightly reduced water intake, resulting in the exact dose consumed being re-calculated as 60 mg/kg (6 mg/kg CBD). Since reduced water intake was unaffected in the dose selection study where the MRX1 formulation contained no terpenes, and the vehicle (Cremophor) likewise had no effect on water intake, the assumption is that the presence of terpenes in both formulation preparations was less palatable to the mice. Calorie intake and weight gain was similar across all three HFD/L-NAME intervention groups.

To confirm that sufficient plasma levels of CBD were being achieved, blood plasma samples were withdrawn at the end of the experimental period and sent for quantitative analysis for CBD and its metabolites 7-OH-CBD (7-hydroxycannabidiol) and 7-COOH-CBD (7-carboxy cannabidiol). Analysis was performed on pooled samples from 5 mice in each group, and 5 independent pooled samples were tested). Referring to FIG. 12, both MRX1 and RGU MRX1 given at the calculated dose of 60 mg/kg achieved similar plasma levels of CBD, 2.6 μM and 2.8 μM, respectively. Both major metabolites of CBD were also detected in similar quantities.

Referring to FIG. 13, HFD/L-NAME resulted in an increase in blood glucose levels, an effect that was not present in either MRX1 treated group. In terms of tissue weights, liver weight was not changed by HFD/L-NAME in any group, whereas all three groups exhibited a marked increase in WAT:BW ratio, as shown in FIG. 13 (top panel). Heart weight was significantly greater in the vehicle treated HFD/L-NAME group compared to normal fed mice, consistent with the development of cardiac hypertrophy. MRX1 significantly prevented this increase in heart weight, while RGU MRX1 achieved a smaller increase in heart weight, although this did not reach statistical significance. Likewise, lung weight was also increased by HFD/L-NAME, an effect that was not seen in mice treated with either MRX1 or RGU MRX1. The wet/dry lung weight ratio was similar across all four groups, as shown in FIG. 13 (bottom panel), indicating that the increase in lung weight in vehicle-treated mice was not due to pulmonary oedema, but instead indicative of pulmonary fibrosis.

As seen in the dose selection study, both MRX1 and RGU MRX1 were seen to have a marked anti-hypertensive effect, reducing both systolic and diastolic blood pressure, as shown in FIG. 14 (top panel). These occurred in the absence of any effect on heart rate, suggesting that the antihypertensive effect is likely mediated via a reduction in peripheral resistance rather than any effect on cardiac output. This was confirmed by pressure volume loop analysis, performed in closed chest animals under terminal anaesthesia, to determine both systolic and diastolic function, where there were no differences in cardiac output across any of the groups, as shown in FIG. 14 (middle panel).

Since this is a model of diastolic dysfunction, changes in systolic function are not marked and ejection fraction (EF) and cardiac contractility (dP/dtmax) was not different between normal fed mice and any of the three HFD/L-NAME groups. However, HFD/L-NAME intervention did increase in end-systolic pressure in the vehicle treated group, as shown in FIG. 14 (middle panel).

In terms of diastolic function, no changes were observed in either ventricular relaxation (dP/dtmin) or left ventricular end-diastolic volume (EDV). However, there was a tendency to an increased left ventricular end-diastolic pressure in the vehicle-treated HFD/L-NAME mice, indicative of stiffening of the ventricular wall, but not in the MRX1 treated groups, although this did not quite reach statistical significance, as shown in FIG. 14 (bottom panel).

Changes in the expression of various markers of heart failure, cardiac fibrosis, and inflammation were determined in heart tissue using qPCR. Referring to FIG. 15, mRNA expression of brain natriuretic peptide (BNP), a hormone secreted by cardiomyocytes in response to stretch caused by increased ventricular blood volume and a clinical marker of heart failure, was markedly increased in response to HFD/L-NAME; this was completely absent in the hearts for mice treated with MRX1 and reduced in mice treated with RGU MRX1. Collagen 1 (Col1) and collagen 3 (Col3) are extracellular matrix proteins whose raised levels are indicative of the presence of cardiac stiffening/fibrosis; both Col1 and Col3 mRNA were significantly raised in the HFpEF model treated with vehicle, an effect that was almost completely suppressed by MRX1, with RGU MRX1 having a smaller protective effect, as shown in FIG. 15. In terms of an inflammatory response, the HFpEF model did not demonstrate any increase in the expression of Nrf2 (master regulator of inflammation), but there was a small (not statistically significant) increase in COX-2 expression; neither MRX1 nor RGU MRX1 altered the expression of either of these two inflammatory markers.

Summary of the intervention study to determine the effects of MRX1 in HfpEF Cannabidiol (CBD), 7-Hydroxycannabidiol (7-OH-CBD), and 7-Carboxy cannabidiol (7-COOH-CBD) were all detected in plasma of mice treated orally for 2 weeks with both MRX1 types given at a dose of 60 mk/kg (6 mg/kg CBD equivalent). The plasma CBD concentration achieved was similar for both preparations (2.6-2.8 μM). At this dose, MRX1 reduced the elevated systolic blood pressure, the increased heart and lung weights, and BNP expression seen in this model of HFpeF. RGU MRX1 also reduced the elevated systolic blood pressure and the increased lung weight.

In this study, the model did not exhibit marked diastolic dysfunction, but there was a trend towards a reduction in EDP in response to MRX1, which may indicate reduced stiffness in the heart muscle. This appears to be linked to an anti-fibrotic effect as seen by a reduced Col1 and Col3 expression in cardiac tissue. Overall, MRX1 exerts anti-hypertensive, anti-hypertrophic, and anti-fibrotic effects.

It is perhaps worthy of note that, in the preliminary dosing study, cytokine analysis was also performed on the WAT, with some interesting findings. For example, in the HFpeF model, adipose tissue expression of IL1ra was markedly increased, and this was dose-dependently reduced by MRX1, as shown in FIG. 16. Although IL1ra is an anti-inflammatory cytokine, it is a biomarker of adipocyte dysfunction and is known to be increased in obesity and metabolic syndrome. Moreover, both JE, an early response gene that encodes the monocyte-specific cytokine MCP-1, and TIMP1, an inhibitory molecule that regulates matrix metalloproteinases and plays a major role in extracellular matrix composition, were highly expressed in WAT from vehicle-treated mice, but not in mice treated with MRX1.

### Example 10—Treatment of Chemotherapy Induced Peripheral Neuropathy (CIPN)

An embodiment of the cannabinoid-based formulation of the invention, which may be referred to herein as MRX1, will be used to treat Chemotherapy Induced Peripheral Neuropathy (CIPN) in an upcoming Phase II placebo controlled double-blind cross-over trial. The Phase II clinical trial will begin in 2024 and will be conducted by the University of Edinburgh. It will be led by Professor Marie Fallon, the St Columba's Hospice Chair of Palliative Medicine and Honorary Consultant in Palliative Care at the Western General Hospital in Edinburgh, Scotland. Professor Fallon is a world-leading research, and has conducted previous clinical trials using CBD.

The Applicant of the present application has signed a Drug Supply Agreement with the University of Edinburgh and the Lothian Health Board on 7 Nov. 2023.

This Phase II trial will assess the efficacy of MRX1 in providing effective analgesia in 92 patients with CIPN, as well as a range of secondary outcomes including motor function, quality of life, anxiety and depression, and sleep quantity and efficiency. The design of the trial is shown in FIG. 17.

The trial will allow the inventors to demonstrate that the cannabinoid-based formulation of the invention, including embodiments such as MRX1, is efficacious in treating CIPN over a period of 5 weeks, as well as improving a range of secondary outcomes.

MRX1 will be administered using an appropriate dosing regimen and titration schedule based on available non-clinical and clinical evidence, as well as data gathered from the Phase I pharmacokinetic study that will be undertaken on MRX1 in 2024, which is discussed further in Example 13.

Data will be gathered on inflammatory markers, to investigate mechanism of action of CBD in the prevention and treatment of CIPN, and dose-response pharmacodynamics using specific and potentially novel biomarkers for CIPN disease state severity, to assist in the evaluation of efficacy and effective dose.

Brain connectome and central nervous system inflammation by fMRI will also be investigated, providing specific and potentially novel biomarkers for CIPN disease state and severity to assist in the evaluation of efficacy and effective dose.

Following the completion of this Phase II trial, the inventors intend to progress MRX1 to a further Phase III trial to demonstrate the efficacy of MRX1 in treating CIPN in a larger population. The inventors also intend to engage with the Medicines and Healthcare Products Regulatory Agency (MHRA) and National Institute for Health and Care Excellence (NICE) to progress the consideration of MRX1 for marketing authorisation to treat CIPN, NICE appraisal, and sale as a licensed drug in the United Kingdom through the NHS.

### Example 11—Treatment of Pain Associated with Endometriosis

An embodiment of the cannabinoid-based formulation of the invention, which may be referred to herein as MRX1, will be used to treat the pain associated with endometriosis in an upcoming Phase II double blind placebo-controlled trial. The Phase II clinical trial will begin in 2024, and will be conducted by the University of Edinburgh. It will be led by Dr Lucy Whitaker, Senior Clinical Research Fellow at the Centre for Reproductive Health, University of Edinburgh.

The Applicant of the present application signed a Drug Supply Agreement with the University of Edinburgh and Lothian Health Board on 22 Feb. 2024.

This Phase II trial will assess the efficacy of MRX1 in treating the pain associated with endometriosis in 100 patients with endometriosis identified at laparoscopy or imaging (performed in the last five years) and chronic pelvic pain of greater than six months duration. The trial will investigate the feasibility of recruitment and retention. It will also assess other symptoms, including fatigue, and the impact on quality of life and number of health care visits.

The design of the trial is a 12-week, double-blind, placebo-controlled trial with no cross-over. It will be conducted across two sites: NHS Lothian and NHS Grampian.

The trial will allow the inventors to demonstrate that MRX1 is efficacious in treating endometriosis associated pain over a period of 12 weeks, as well as improving a range of secondary outcomes outlined above. It will also record patient use of opioids before and during the treatment period to gather data on the ability of MRX1 to reduce opioid use.

MRX1 will be administered using an appropriate dosing regimen and titration schedule based on available non-clinical and clinical evidence, as well as data gathered from a Phase I pharmacokinetic study that will be undertaken on MRX1 in 2024, which is discussed further in Example 13.

Blood samples will be taken for testing of inflammatory markers to investigate mechanism of action of CBD in the prevention and treatment of pain associated with endometriosis.

Following the completion of this Phase II trial, the inventors intend to progress MRX1 to a further Phase III trial to demonstrate the efficacy of MRX1 in treating pain associated with endometriosis in a larger population. They also intend to engage with the Medicines and Healthcare Products Regulatory Agency (MHRA) and National Institute for Health and Care Excellence (NICE) to progress the consideration of MRX1 for marketing authorisation to treat endometriosis, NICE appraisal, and sale as a licensed drug in the United Kingdom through the NHS.

### Example 12—Resolution of Pathogenic Nature of Fibroblasts and Mesenchymal Stromal Cells in Endometriosis

Fibroblasts play a significant role in endometriosis, contributing to its complex pathogenesis. Briefly, fibroblasts affect the remodelling of the extracellular matrix (ECM), contribute to inflammatory processes, modulate angiogenesis, and are known to be immunomodulatory. All these processes are involved in endometriosis, and there is evidence of fibroblast involvement in all these processes.

Utilising an embodiment of the cannabinoid-based formulation of the invention, which may be referred to herein as MRX1, a co-funded PhD studentship at the University of Reading under the supervision of Professor Darius Widera will perform, and investigate, the following:

1) Inflammation will be simulated by exposing fibroblasts/mesenchymal stem/stromal cells (MSCs) to Tumour Necrosis Factor-α, IL-1β, and IL-6, either alone, or in combination with MRX1, followed by immunocytochemical staining against the NF-κB subunit p65. Imaging and analysis of NF-κB translocation will be performed on a Revvity Operetta high-content imaging system (with machine learning capacity).

2) Secretion of pro-angiogenic and pro-inflammatory factors will be assessed by ELISA (e.g., FGF-2, IL-6, IL-1β, VEGF).

3) Secretomes from fibroblasts/MSCs treated as described in 2) will be collected and used in a HUVEC in vitro angiogenesis model.

4) Secretomes generated as above will be used in a human monocyte polarisation assay.

5) Fibroblast and MSCs will be treated as described in 1), and the composition of the deposited ECM (Collagens, Proteoglycans, and Glycoproteins (e.g., fibronectin, laminin), Matrix Metalloproteinases (MMPs), Integrins, and Basement Membrane Components) will be studied using immunocytochemistry and high-content imaging (see 1 above).

The inventors hypothesise that MRX1 can reduce inflammatory signalling, production of pro-angiogenic factors, and ECM remodelling by fibroblasts and MSCs, and modulate immune cells towards a regulatory M2-phenotype.

### Example 13—Phase I Pharmacokinetic (PK) Study

An embodiment of the cannabinoid-based formulation of the invention, which may be referred to herein as MRX1, will be administered to healthy volunteers to determine the safety and PK profile of the MRX1 formulation. MRX1 will be administered to between 20 and 30 participants using a dosing regimen and titration schedule aligned with non-clinical and clinical evidence, and the inventors' expected administration of the MRX1 formulation in clinical practice to treat various chronic inflammatory pain conditions.

The Phase I PK study will be conducted in 2024 by Galeno Research unit in Brazil. MRX1 will be administered to participants over a period of 1-3 weeks. Blood samples will be taken at regular intervals, and those blood samples will be tested for analytes including: Cannabidiol (CBD), 7-Carboxy cannabidiol (7-COOH-CBD), 7-Hydroxy cannabidiol (7-OH-CBD), Delta-9 Tetrahydrocannabinol (THC), 11-carboxy-Δ9-THC (11-OH-THC), and 11-hydroxy-Δ9-THC (11-COOH-THC).

In line with the novel and innovative characteristics of the cannabinoid-based formulation of the invention, and the fact that the formulation substantially lacks THC, the inventors expect that there will be a low or zero level of Delta-9 Tetrahydrocannabinol (THC) and metabolites detected in the blood samples of participants. This would provide further evidence of the novelty of the cannabinoid-based formulation of the invention when administered to humans.

Based on their research and the results of the permeability study previously conducted on the cannabinoid-based formulation of the invention, as discussed in Example 7, the inventors also expect that the cannabinoid-based formulation of the invention, such as MRX1, will deliver superior CBD bioavailability to that of other CBD in oil formulations. The presence of the proprietary terpene blend of the cannabinoid-based formulation of the invention is expected to increase permeability, and therefore bioavailability, of CBD in the blood.

Following the completion of this Phase I trial, the inventors will be able to utilise these proven novel characteristics, and the bioavailability data which is generated, to better target the dosing regimen and titration schedule used for the cannabinoid-based formulation of the invention in additional clinical trials, including two Phase II clinical trials being undertaken by the University of Edinburgh using the cannabinoid-based formulation of the invention to treat the pain associated with chemotherapy induced peripheral neuropathy and endometriosis, respectively, as discussed in Examples 10 and 11.

### CONCLUSIONS

The resultant CBD formulations are referred to as Cannabis-Based Products for Medicinal use (CBPM) in humans or animals. In particular, the CBPMs are formulations containing cannabidiol (CBD) isolate, terpenes and medium-chain triglyceride (MCT) oil in a liquid form for sublabial, buccal, sublingual, or oropharyngeal administration delivery. The use of these formulations focuses primarily on conditions associated with pain, inflammation, autoimmune diseases, neurological diseases, neoplasm, nausea, hypertension, hypertrophic cardiomyopathy, and fibrosis.

The formulations (or CBPMs) are broad-spectrum CBD mixtures, which consist of a particular combination of CBD and terpenes, produced at pharmaceutical standards under the guidelines of Good Manufacturing Practice (GMP) and designed to produce precise physical effects. To date, there are no products available in the UK that comply with the GMP standard, and so the pharmaceutical formulations of the invention address this gap and facilitate the access and delivery of a pharmaceutical standard CBPM for use in clinical trials and for treating patients.

The preparation of these liquid formulations for sublabial, buccal or sublingual delivery allows the introduction of the drug into the systemic circulation via a mucous membrane, increasing the onset of activity and potency due to bypassing first-pass metabolism and increasing the drug bioavailability. Advantageously, given the lipophilicity and poor bioavailability of oral administration of cannabinoids for gastrointestinal absorption, which is considered the most popular method currently in the market for the delivery of medicines, the formulations of the invention surprisingly enable administration by this route.

The active pharmaceutical components of the CBPM formulations are the CBD and terpenes. The MCT oil is acting as a pharmaceutically acceptable carrier, excipient or diluent and is, therefore, not considered to be an active component. The active components are believed to interact with the endocannabinoid system (ECS) and modulate a plethora of physiological responses. The ECS is a complex lipid cell-signalling system comprised of the cannabinoid receptors (CBRs) 1 and 2, the endogenous cannabinoids (endocannabinoids), anandamide (N-arachidonoylethanolamide, AEA) and 2-Arachidonoylglycerol (2-AG), the anandamide transporter protein (TP), and the enzymes responsible for the synthesis and degradation of endocannabinoids (fatty acid amide hydrolase, FAAH; and monoacylglycerol lipase, MAGL). However, further research into the endocannabinoid system pharmacology has led to the discovery of a variety of molecular targets for cannabinoids and terpenes independent of the CBRs 1 and 2, such as the transient receptor potential (TRP) channels, two orphan G-protein coupled receptors (GPR55 and GPCR18) and the peroxisome proliferator-activated receptor (PPAR). The ECS regulates homeostasis in various physiological processes such as inflammation, pain, appetite, metabolism, and memory.

The ECS orchestrates a ‘retrograde negative feedback mechanism’ in the CNS. Upon neuronal depolarisation, AEA and 2-AG are synthesised on the postsynaptic terminal in dendritic spines and somatodendritic compartments. They are then released into the neuronal cleft to suppress the inhibitory neurotransmitter gamma-aminobutyric acid (GABA) secretion in GABAergic afferents and the excitatory neurotransmitter glutamate in glutamatergic neurons. Additionally, eCB signalling in non-neuronal tissues regulates several physiological processes, such as spermatogenesis, pain and the modulation of the immune system. Given that the ECS modulates multiple cell functions, which play critical roles in pain, neuromodulation, mood, immunity and other physiological processes, therapeutics targeting this system, such as the CBPMs described herein, hold potential as novel approaches in treating various conditions, such as pain, inflammation, autoimmune disease, neurological disease, neoplasm, nausea, hypertension, hypertrophic cardiomyopathy, and fibrosis.

CBPMs have been added to the treatment paradigm for many patients suffering from acute and chronic inflammatory conditions, pain, and symptoms of negative affect such as anxiety and depression that have limited or a lack of response to traditional therapeutic approaches. Due to this, the National Institute for Health and Care Excellence (NICE) and the International Association for the Study of Pain (IASP) have made specific calls for research using CBPMs in humans.

Therefore, there is a current unmet need for treating multiple treatment-resistant conditions, such as for use in treating pain, inflammation, autoimmune disease, neurological disease, neoplasm, nausea, hypertension, hypertrophic cardiomyopathy, and fibrosis. Since no one-size-fits-all therapy is available, most patients require multiple and alternative treatment approaches. Additionally, numerous CBD formulations are now available as food supplements, making many beneficial health claims, with minimal assessment of whether they are being administered in a suitable formulation even to achieve blood levels to support these claims. The method of the invention addresses this need, and produces a medical quality CBPM at pharmaceutical standards and under the guidelines of GMP aimed for its use in medical research and practice.

1. A cannabinoid-based formulation comprising:
(i) one or more cannabinoid; and
(ii) a plurality of terpenes selected from a group of terpenes consisting of: myrcene; caryophyllene; ocimene; pinene; limonene; humulene; and linalool;
wherein the formulation substantially lacks THC.

2. The cannabinoid-based formulation according to claim 1, wherein the concentration of THC in the formulation is less than 0.01% (w/v), less than 0.005% (w/v), or less than 0.001% (w/v).

3. The cannabinoid-based formulation according to either claim 1 or claim 2, wherein the concentration of THC in the formulation is less 0.0005% (w/v), less than 0.0002% (w/v), or less than 0.0001% (w/v).

4. The cannabinoid-based formulation according to any preceding claim, wherein the formulation comprises no detectable THC using reasonable means of analysis.

5. The cannabinoid-based formulation according to any preceding claim, wherein the one or more cannabinoid is selected from a group of cannabinoids consisting of: cannabidiol (CBD); cannabichromene (CBC); cannabigerol (CBG); and cannabigerol monomethyl ether (CBGM).

6. The cannabinoid-based formulation according to any preceding claim, wherein the one or more cannabinoid comprises CBD.

7. The cannabinoid-based formulation according to any preceding claim, wherein the one or more cannabinoid comprises an isolate of cannabidiol (CBD).

8. The cannabinoid-based formulation according to any preceding claim, wherein the formulation does not comprise CBC, CBG, or CBGM.

9. The cannabinoid-based formulation according to any preceding claim, wherein the purity of the one or more cannabinoid is at least 97%, at least 98% or at least 99%, or about 98-100%.

10. The cannabinoid-based formulation according to any preceding claim, wherein the concentration of the one or more cannabinoid, preferably CBD, in the cannabinoid-based formulation is at least 2% (w/v), at least 3% (w/v) or at least 4% (w/v).

11. The cannabinoid-based formulation according to any preceding claim, wherein the concentration of the one or more cannabinoid, preferably CBD, in the cannabinoid-based formulation is at least 5% (w/v), at least 6% (w/v) or at least 7% (w/v).

12. The cannabinoid-based formulation according to any preceding claim, wherein the concentration of the one or more cannabinoid, preferably CBD, in the cannabinoid-based formulation is at least 8% (w/v), at least 9% (w/v) or at least 10% (w/v).

13. The cannabinoid-based formulation according to any preceding claim, wherein the concentration of the one or more cannabinoid, preferably CBD, in the cannabinoid-based formulation is between 7 and 13% (w/v), or between 8 and 12% (w/v).

14. The cannabinoid-based formulation according to any preceding claim, wherein the concentration of the one or more cannabinoid, preferably CBD, in the cannabinoid-based formulation is between 9 and 11% (w/v).

15. The cannabinoid-based formulation according to any preceding claim, wherein the purity of the plurality of terpenes may be at least 90%, at least 92% or at least 95% of total terpenes.

16. The cannabinoid-based formulation according to any preceding claim, wherein the plurality of terpenes comprises at least three, at least four, or at least five terpenes selected from a group of terpenes consisting of: myrcene; caryophyllene; ocimene; pinene; limonene; humulene; and linalool.

17. The cannabinoid-based formulation according to any preceding claim, wherein the plurality of terpenes comprises at least six, or at least seven terpenes selected from a group of terpenes consisting of: myrcene; caryophyllene; ocimene; pinene; limonene; humulene; and linalool.

18. The cannabinoid-based formulation according to any preceding claim, wherein:
(i) the pinene is α-Pinene, optionally wherein the concentration of α-Pinene in the cannabinoid-based formulation is at least 0.0001 (w/v), at least 0.001% (w/v), at least 0.01% (w/v), less than 0.75% (w/v), less than 0.5% (w/v), less than 0.3% (w/v), between 0.0001 and 0.5% (w/v), between 0.01 and 0.3% (w/v), between 0.01 and 0.2% (w/v), between 0.01 and 0.05% (w/v), between 0.01 and 0.04% (w/v), or between 0.01 and 0.03% (w/v);
(ii) the concentration of limonene in the cannabinoid-based formulation is at least 0.0001 (w/v), at least 0.001% (w/v), at least 0.01% (w/v), less than 0.75% (w/v), less than 0.5% (w/v), less than 0.3% (w/v), between 0.0001 and 0.5% (w/v), between 0.01 and 0.3% (w/v), between 0.01 and 0.2% (w/v), between 0.01 and 0.05% (w/v), between 0.01 and 0.04% (w/v), between 0.01 and 0.03% (w/v), between 0.03 and 0.09% (w/v), between 0.03 and 0.08% (w/v), or between 0.03 and 0.07% (w/v);
(iii) the ocimene is β-Ocimene, optionally wherein the concentration of β-Ocimene in the cannabinoid-based formulation is at least 0.0001 (w/v), at least 0.001% (w/v), at least 0.01% (w/v), less than 0.75% (w/v), less than 0.5% (w/v), less than 0.3% (w/v), between 0.0001 and 0.5% (w/v), between 0.01 and 0.3% (w/v), between 0.01 and 0.2% (w/v), between 0.01 and 0.07% (w/v), between 0.01 and 0.06% (w/v), between 0.01 and 0.05% (w/v), between 0.01 and 0.04% (w/v), between 0.001 and 0.05% (w/v), between 0.001 and 0.04% (w/v), between 0.001 and 0.02% (w/v), or between 0.001 and 0.01% (w/v);
(iv) the caryophyllene is β-Caryophyllene, optionally wherein the concentration of β-Caryophyllene in the cannabinoid-based formulation is at least 0.0001 (w/v), at least 0.001% (w/v), at least 0.01% (w/v), less than 0.75% (w/v), less than 0.5% (w/v), less than 0.3% (w/v), between 0.0001 and 0.5% (w/v), between 0.01 and 0.3% (w/v), between 0.01 and 0.2% (w/v), between 0.01 and 0.08% (w/v), between 0.01 and 0.07% (w/v), between 0.01 and 0.06% (w/v), between 0.01 and 0.05% (w/v), between 0.01 and 0.1% (w/v), between 0.01 and 0.09% (w/v), between 0.01 and 0.08% (w/v), or between 0.03 and 0.08% (w/v);
(v) the humulene is Alpha-Humulene, optionally wherein the concentration of Alpha-Humulene in the cannabinoid-based formulation is at least 0.0001 (w/v), at least 0.001% (w/v), at least 0.01% (w/v), less than 0.75% (w/v), less than 0.5% (w/v), less than 0.3% (w/v), between 0.0001 and 0.5% (w/v), between 0.01 and 0.3% (w/v), between 0.01 and 0.2% (w/v), between 0.01 and 0.04% (w/v), between 0.01 and 0.03% (w/v), between 0.01 and 0.02% (w/v), between 0.005 and 0.04% (w/v), between 0.005 and 0.03% (w/v) or between 0.005 and 0.02% (w/v);
(vi) the myrcene is β-Myrcene, optionally wherein the concentration of β-Myrcene in the cannabinoid-based formulation is at least 0.0001 (w/v), at least 0.001% (w/v), at least 0.01% (w/v), less than 0.75% (w/v), less than 0.5% (w/v), less than 0.3% (w/v), between 0.0001 and 0.5% (w/v), between 0.01 and 0.3% (w/v), between 0.01 and 0.2% (w/v), between 0.01 and 0.1% (w/v), between 0.01 and 0.08% (w/v), between 0.01 and 0.07% (w/v), or between 0.01 and 0.06% (w/v); and/or
(vii) the concentration of linalool in the cannabinoid-based formulation is at least 0.0001 (w/v), at least 0.001% (w/v), at least 0.01% (w/v), less than 0.75% (w/v), less than 0.5% (w/v), less than 0.3% (w/v), between 0.0001 and 0.5% (w/v), between 0.01 and 0.3% (w/v), between 0.01 and 0.2% (w/v), between 0.01 and 0.05% (w/v), between 0.01 and 0.04% (w/v), between 0.01 and 0.03% (w/v), or between 0.01 and 0.02% (w/v).

19. The cannabinoid-based formulation according to any preceding claim, wherein the cannabinoid-based formulation further comprises a pharmaceutically acceptable excipient or carrier.

20. The cannabinoid-based formulation according to claim 19, wherein the pharmaceutically acceptable excipient or carrier comprises medium-chain triglyceride (MCT), preferably MCT oil.

21. The cannabinoid-based formulation according to claim 20, wherein the chemical formula of the MCT is shown below: 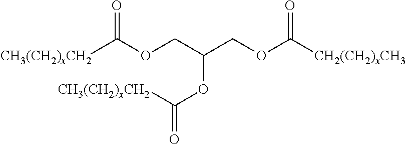
wherein X=1-14.

22. The cannabinoid-based formulation according to either claim 20 or claim 21, wherein the concentration of MCT oil in the cannabinoid-based formulation is at least 80% (w/v), at least 82% (w/v), at least 84% (w/v), at least 86% (w/v), at least 88% (w/v), less than 96% (w/v), less than 95% (w/v) less than 94% (w/v), less than 92% (w/v), less than 90% (w/v), between 85 and 93% (w/v), between 86 and 92% (w/v), between 87 and 91% (w/v), or between 88 and 90% (w/v).

23. The cannabinoid-based formulation according to any preceding claim, wherein the cannabinoid-based formulation is in a liquid form, and optionally comprises a tincture.

24. The cannabinoid-based formulation according to any preceding claim, wherein the formulation is administrable by sublabial, buccal, sublingual, or oropharyngeal administration delivery.

25. A method of producing the cannabinoid-based formulation according to any one of claims 1 to 24, the method comprising combining: (i) one or more cannabinoid; and (ii) a plurality of terpenes selected from a group of terpenes consisting of: myrcene; caryophyllene; ocimene; pinene; limonene; humulene; and linalool, to produce a cannabinoid-based formulation, wherein the formulation substantially lacks THC.

26. A cannabinoid-based formulation obtained or obtainable by the method according to claim 25.

27. The cannabinoid-based formulation according to any one of claims 1 to 24 or 26, for use in therapy.

28. The cannabinoid-based formulation according to any one of claims 1 to 24 or 26, for use in the treatment, amelioration or prevention of pain, inflammation, an autoimmune disease, a neurological disease, neoplasm, nausea, hypertension, hypertrophic cardiomyopathy, or fibrosis.

29. The cannabinoid-based formulation for use according to claim 28, wherein the pain, inflammation, autoimmune disease, neurological disease, neoplasm, nausea, hypertension, hypertrophic cardiomyopathy, or fibrosis, are selected from the group of conditions consisting of: chemotherapy-induced peripheral neuropathy (CIPN), endometriosis, osteoarthritis, rheumatoid arthritis, ulcerative colitis, Crohn's disease, fibromyalgia, irritable bowel syndrome, asthma, chronic obstructive pulmonary disease (COPD), gout, scleroderma, lupus, Ehlers-Danlos syndrome, pericarditis, myocarditis, myocardial infarction, chemotherapy-induced nausea, anxiety, Type I diabetes, cardiovascular disease, primary hypertension, secondary hypertension, resistant hypertension, isolated systolic hypertension, malignant hypertension, obstructive hypertrophic cardiomyopathy, nonobstructive hypertrophic cardiomyopathy, pulmonary fibrosis, liver fibrosis, heart fibrosis, kidney fibrosis, mediastinal fibrosis, retroperitoneal cavity fibrosis, bone marrow fibrosis, skin fibrosis, and scleroderma.
